(19) United States
(12) Patent Application Publication
(10) Pub. No.: US 2025/0054568 A1
(43) Pub. Date: Feb. 13, 2025

(54) Computational Method To Identify Gene Networks Containing Functionally-Related Genes

(71) Applicant: LifeMine Therapeutics, Inc., Cambridge, MA (US)

(72) Inventors: Saheed IMAM, Cambridge, MA (US); Michalis HADJITHOMAS, Cambridge, MA (US)

(21) Appl. No.: 18/746,664

(22) Filed: Jun. 18, 2024

Related U.S. Application Data
(63) Continuation of application No. PCT/US2023/060166, filed on Jan. 5, 2023.
(60) Provisional application No. 63/297,565, filed on Jan. 7, 2022.

(51) Int. Cl. G16B 5/00; G06F 30/27; G16B 40/20

(52) U.S. Cl. CPC G16B 5/00; G06F 30/27; G16B 40/20

(57) ABSTRACT

Computational methods for identification of biosynthetic gene clusters (BGCs) that is independent of prior identification of a core synthase are described. The disclosed methods also facilitate linking BGCs to their potential downstream targets. The approach integrates information from co-evolution, co-occurrence, co-regulation, co-localization, and functional enrichment to group functionally-related genes across a gene network, delineate BGCs, and propose targets for the putative secondary metabolites.

### CROSS-REFERENCE TO RELATED APPLICATIONS

This application is a continuation of International Application No. PCT/US2023/060166, which designated the United States and was filed on Jan. 5, 2023, published in English, which claims the priority benefit of U.S. Provisional Patent Application Ser. No. 63/297,565, filed Jan. 7, 2022, the contents of which are incorporated herein by reference in their entirety.

### FIELD

The present disclosure relates generally to methods and systems for identifying genes associated with gene networks, e.g., biosynthetic gene clusters, and applications thereof, including identifying potential therapeutic targets and drug candidates.

### BACKGROUND

Secondary metabolites identified from bacteria, fungi and plants have found use in wide variety of applications in medicine and agriculture (see, e.g., Katz, et al. (2016), “Natural Product Discovery: Past, Present, and Future”, J Ind Microbiol Biotechnol. 43(2-3):155-76). These secondary metabolites are often synthetized via metabolic pathways consisting of core biosynthetic enzymes (such as polyketide synthases and non-ribosomal peptide synthases) and a variety of tailoring enzymes, which are colocalized on the genome as biosynthetic gene clusters (BGCs) (see, e.g., Scherlach, et al. (2021), “Mining and Unearthing Hidden Biosynthetic Potential”, Nat Commun. 12(1):3864). With the availability of vast numbers of sequenced genomes, genomics database mining techniques have become crucial for the discovery of networks of functionally-related genes, including BGCs. Traditional approaches for identifying the BGCs have relied on identification of core synthases using profile hidden Markov models and subsequent inclusion of genes proximal to these core enzymes in the genome (see, for example, Blin, et al. (2021), “antiSMASH 6.0: Improving Cluster Detection and Comparison Capabilities”, Nucleic Acids Res. 49(W1):W29-W35; Scherlach, et al. (2021), ibid.). However, there are several drawbacks to such approaches. Firstly, not all BGCs contain core enzymes, as many secondary metabolites can be synthesized from backbones derived from central metabolism (see, e.g., Wasil, et al. (2018), “Oryzines A & B, Maleidride Congeners from Aspergillus oryzae and Their Putative Biosynthesis”, J. Fungi 4:96; Lim, et al. (2018), “Fungal Isocyanide Synthases and Xanthocillin Biosynthesis in Aspergillus fumigatus”, mBio 9(3):e00785-18.). Currently, ˜11% of the experimentally verified BGCs in the MiBiG database (see, e.g., Kautsar, et al. (2020), “MIBiG 2.0: A Repository for Biosynthetic Gene Clusters of Known Function”, Nucleic Acids Res. 48(D1):D454-D458) are classified as “Other”, i.e., BGCs not having an associated core synthase. Secondly, these approaches are dependent on co-localization of the genes for the core synthase and associated tailoring enzymes, potentially limiting the scope of metabolic interactions that could be involved in the synthesis of secondary metabolites. Finally, a major challenge in genome mining-based BGC identification is linking the identified BGCs with the potential downstream targets of the synthesized secondary metabolite. One approach to making this connection is the embedded resistance gene hypothesis (Yan, et al. (2020), “Recent Developments in Self-Resistance Gene Directed Natural Product Discovery”, Nat Prod Rep. 37(7):879-892), wherein the gene encoding the protein target of the secondary metabolite undergoes a duplication event, with a paralogous copy becoming embedded within the BGC and undergoing accelerated evolution thereby resulting in acquisition of mutations that nullify the impact of the secondary metabolite without impacting the primary protein function. While this hypothesis has been verified through genomic analysis of the BGCs associated with previously discovered secondary metabolites, and has also proven powerful for identification of novel targets (Yan, et al. (2020), ibid.), it limits the identification of resistance genes to those embedded within the BGC.

### BRIEF SUMMARY OF THE INVENTION

To circumvent the shortcomings outlined above, a computational approach for the identification of networks of functionally-related genes is described. In some instances, the computational approach may be used to identify, e.g., BGCs independently of an association with a core synthase, and may also facilitate linking BGCs to their potential downstream targets. The approach integrates information from co-evolution, co-occurrence, co-regulation, co-localization, and functional enrichment to: (i) group functionally-related genes across a gene network, (ii) delineate a specific type of gene network (e.g., a BGC), and (iii) propose targets for the associated secondary metabolites in the case that the methods are used to identify BGCs. The disclosed methods (and systems designed to implement the disclosed methods) are based on the observation that functionally related genes co-evolve (Steenwyk, et al. (2022), “An Orthologous Gene Coevolution Network Provides Insight Into Eukaryotic Cellular and Genomic Structure and Function”, Sci. Adv. 8, eabn0105) and are often co-regulated. Furthermore, as previously stated, genes within BGCs are often co-localized and co-occur in related organisms. The disclosed methods provide a pipeline for gene network (e.g., BGC) discovery and functional assignment.

Disclosed herein are computer-implemented methods for identification of networks of functionally-related genes, the method comprising: receiving a selection of genomes for analysis as input, wherein the selection of genomes comprises a plurality of related genomes; identifying clusters of orthologous genes (COGs) in the plurality of related genomes; determining a pairwise co-evolution metric, a pairwise co-regulation metric, a pairwise co-occurrence metric, a pairwise co-localization metric, or any combination thereof, for the identified COGs; determining pairwise functional association scores for the identified COGs based on the determined pairwise co-evolution metrics, pairwise co-regulation metrics, pairwise co-occurrence metrics, pairwise co-localization metrics, or any combination thereof; clustering the identified COGs according to their pairwise functional association scores to group functionally-related COGs; and outputting, based on a functional enrichment analysis performed on at least one COG cluster to identify COG clusters that are enriched for genes in a specific functional category, a determination that a COG cluster is a network of functionally-related genes in the specific functional category.

In some embodiments, the functional enrichment analysis does not require identification of a gene known to be associated with gene networks of a specific functional category.

In some embodiments, the functional enrichment analysis comprises testing for enrichment of genes in functional categories known to be associated with biosynthetic gene clusters (BGCs), thereby identifying those COG clusters as putative BGCs. In some embodiments, the functional categories known to be associated with BGCs comprise gene ontology terms or KEGG pathways known to be associated with BGCs. In some embodiments, the gene ontology terms known to be associated with BGCs comprise GO:0019748 (secondary metabolic process), GO:0044550 (secondary metabolite biosynthetic process), GO:0030639 (polyketide biosynthetic process), GO:0030638 (polyketide metabolic process), GO:0043455 (regulation of secondary metabolic process), GO:1900539 (fumonisin metabolic process), or any combination thereof. In some embodiments, the KEGG pathways known to be associated with BGCs comprise M00778 (Type II polyketide backbone biosynthesis) or M00095 (C5 isoprenoid biosynthesis, mevalonate pathway), M00937 (Aflatoxin biosynthesis), M00893 (Lovastatin biosynthesis), or any combination thereof.

In some embodiments, the functional enrichment analysis comprises testing for enrichment of protein domain representations known to be associated with biosynthetic gene clusters (BGCs), thereby identifying those COG clusters as putative BGCs. In some embodiments, the protein domain representations known to be associated with BGCs comprise PFAM domain representations, Conserved Domain Database (CDD) domain representations, or TIGRFAM domain representations known to be associated with BGCs.

In some embodiments, the computer-implemented method further comprises identifying putative targets for a secondary metabolite synthesized by a putative BGC by: identifying protein sequences that are not components of known BGCs; determining pairwise functional association scores for the identified protein sequences and the putative BGC; and identifying putative targets for the secondary metabolite based on a comparison of the pairwise functional association scores to a first predetermined threshold. In some embodiments, the pairwise functional associate score comprises a co-regulation score, and the first predetermined threshold for the co-regulation score corresponds to a p-value of less than or equal to 0.05. In some embodiments, the pairwise functional association score comprises a co-evolution score, and the first predetermined threshold for the co-evolution score corresponds to a co-evolution score value of greater than or equal to 0.7. In some embodiments, the pairwise functional association score comprises a co-occurrence score, and the first predetermined threshold for the co-occurrence score corresponds to a co-occurrence score of greater than or equal to 0.5.

In some embodiments, identification of a putative BGC does not require identification of an associated core synthase.

In some embodiments, the plurality of related genomes comprise fungal, bacterial, or plant genomes.

In some embodiments, identifying COGs comprises identifying orthologous genes in the plurality of related genomes as bidirectional best hits using BLAST, followed by clustering of the identified orthologous genes. In some embodiments, identifying COGs comprises identifying orthologous genes in the plurality of related genomes using orthoMCL or orthoFinder.

In some embodiments, determining a pairwise co-evolution metric for COGs comprises: computing a percent identity between each pair of protein sequences within each COG of a pair of COGs to identify shared protein sequences; computing a Pearson's correlation coefficient for each pair of COGs that include a specified minimum number of shared protein sequences to estimate a rate of co-evolution; filtering the COGs by excluding COGs for which the pairwise Pearson's correlation coefficient is less than a second predetermined threshold and clustering the remaining COGs according to the estimated rates of co-evolution; and performing a functional enrichment analysis to exclude clusters of COGS that are enriched for essential metabolic functional categories. In some embodiments, the second predetermined threshold corresponds to a Pearson's correlation coefficient value of 0.7, 0.8, 0.9, 0.95, 0.98, or 0.99. In some embodiments, clustering the remaining COGs according to the estimated rates of co-evolution comprises use of a Markov clustering (MCL) or hierarchical clustering algorithm.

In some embodiments, determining a pairwise co-regulation metric for COGs comprises: extracting intergenic regions within each COG; performing a de novo detection of sequence motifs within the extracted intergenic regions to identify putative cis-regulatory elements or transcription factor binding sites (TFBS); comparing the putative cis-regulatory elements or TFBS identified for each COG to those identified across all other COGs to determine pairwise motif similarity scores between COGs; filtering the COGs to exclude COGs for which pairwise motif similarity scores have a p-value of less than or equal to a third predetermined threshold; and clustering the filtered COGs based on the pairwise motif similarity scores to identify co-regulated COG clusters. In some embodiments, the third predetermined threshold corresponds to a p-value of 0.05. In some embodiments, the third predetermined threshold corresponds to a p-value of 0.01. In some embodiments, clustering the remaining COGs according to the motif similarity scores comprises use of a Markov clustering (MCL) or hierarchical clustering algorithm.

In some embodiments, determining a pairwise co-occurrence metric for COGs comprises: computing a Jaccard index for each pair of COGs (COG A and COG B) based on a relationship:

Co-occurrence score=(|A∩B|)/(|A∪B|)

filtering the COGs to exclude COGs for which pairwise co-occurrence scores have a value of less than a fourth predetermined threshold; and clustering the filtered COGs based on the pairwise co-occurrence scores to identify co-occurring COG clusters. In some embodiments, the fourth predetermined threshold corresponds to a pairwise co-occurrence score value of 0.3, 0.4, 0.5, 0.6, 0.7, 0.8, or 0.9. In some embodiments, clustering the remaining COGs according to the co-occurrence scores comprises use of a Markov clustering (MCL) or hierarchical clustering algorithm.

In some embodiments, determining a pairwise co-localization metric for COGs comprises: computing a proximity score for each pair of corresponding gene sequences in a pair of COGS (COG A and COG B) based on a relationship:

$$Proximity score = 1 / ( 1 + \# of interspacing genes$$

averaging to determine the co-localization metric for each pair of COGs; and clustering the COGs based on the averaged proximity score to identify co-localized COG clusters. In some embodiments, clustering of COGs according to the averaged proximity score comprises use of a Markov clustering (MCL) or hierarchical clustering algorithm. In some embodiments, the clustering is performed using a Markov clustering (MCL) algorithm and an MCL inflation value ranging from 1.5 to 5.0.

In some embodiments, the pairwise functional association score for the identified COGs is based on addition of the determined pairwise co-evolution metrics, pairwise co-regulation metrics, pairwise co-occurrence metrics, pairwise co-localization metrics, or any combination thereof.

In some embodiments, clustering the identified COGs according to their pairwise functional association scores comprises the use of a use of a Markov clustering (MCL) or hierarchical clustering algorithm.

In some embodiments, the computer-implemented method further comprises determining a horizontal gene transfer metric based on computing a codon adaptation index (CAI) or a dinucleotide signature dissimilarity index (DSDI). In some embodiments, the horizontal gene transfer metric is used to further refine clustering of co-localized, co-occurring and/or co-evolving COGs. In some embodiments, the horizontal gene transfer metric is used as part of a post-processing step to retrieve nearby horizontally transferred genes missed in upstream clustering steps.

In some embodiments, the computer-implemented method further comprises evaluating a gene identified as belonging to the putative BGC to determine if it is a resistance gene. In some embodiments, the resistance gene is an embedded target gene (ETaG) or a non-embedded target gene (NETaG). In some embodiments, the computer-implemented method further comprises performing an in vitro assay to test a secondary metabolite produced by the putative BGC for activity against a resistance gene homolog, or protein encoded thereby, identified in a target genome. In some embodiments, the computer-implemented method further comprises performing an in vivo assay to test a secondary metabolite produced by the putative BGC for activity against a resistance gene homolog, or protein encoded thereby, identified in a target genome. In some embodiments, the target genome comprises a mammalian genome, a human genome, an avian genome, a reptilian genome, an amphibian genome, a plant genome, a fungal genome, a bacterial genome, or a viral genome.

Also disclosed herein are systems comprising: one or more processors; and a memory communicatively coupled to the one or more processors and configured to store instructions that, when executed by the one or more processors, cause the system to: receive a selection of genomes for analysis as input, wherein the selection of genomes comprises a plurality of related genomes; identify clusters of orthologous genes (COGs) in the plurality of related genomes; determine a pairwise co-evolution metric, a pairwise co-regulation metric, a pairwise co-occurrence metric, a pairwise co-localization metric, or any combination thereof, for the identified COGs; determine pairwise functional association scores for the identified COGs based on the determined pairwise co-evolution metrics, pairwise co-regulation metrics, pairwise co-occurrence metrics, pairwise co-localization metrics, or any combination thereof; cluster the identified COGs according to their pairwise functional association scores to group functionally-related COGs; and output, based on a functional enrichment analysis performed on at least one COG cluster to identify COG clusters that are enriched for genes in a specific functional category, a determination that a COG cluster is a network of functionally-related genes in the specific functional category.

In some embodiments, the functional enrichment analysis does not require identification of a gene known to be associated with gene networks of a specific functional category. In some embodiments, the functional enrichment analysis comprises testing for enrichment of genes in functional categories known to be associated with biosynthetic gene clusters (BGCs), thereby identifying those COG clusters as putative BGCs. In some embodiments, the functional categories known to be associated with BGCs comprise gene ontology terms or KEGG pathways known to be associated with BGCs. In some embodiments, the functional enrichment analysis comprises testing for enrichment of protein domain representations known to be associated with biosynthetic gene clusters (BGCs), thereby identifying those COG clusters as putative BGCs. In some embodiments, the protein domain representations known to be associated with BGCs comprise PFAM domain representations, Conserved Domain Database (CDD) domain representations, or TIGRFAM domain representations known to be associated with BGCs.

In some embodiments, the system further comprises instructions for identifying putative targets for a secondary metabolite synthesized by a putative BGC by: identifying protein sequences that are not components of known BGCs; determining pairwise functional association scores for the identified protein sequences and the putative BGC; and identifying putative targets for the secondary metabolite based on a comparison of the pairwise functional association scores to a first predetermined threshold. In some embodiments, the pairwise functional associate score comprises a co-regulation score, and the first predetermined threshold for the co-regulation score corresponds to a p-value of less than or equal to 0.05. In some embodiments, the pairwise functional association score comprises a co-evolution score, and the first predetermined threshold for the co-evolution score corresponds to a co-evolution score value of greater than or equal to 0.7. In some embodiments, the pairwise functional association score comprises a co-occurrence score, and the first predetermined threshold for the co-occurrence score corresponds to a co-occurrence score of greater than or equal to 0.5.

In some embodiments, identification of a putative BGC does not require identification of an associated core synthase.

Disclosed herein are non-transitory computer-readable media storing one or more programs, the one or more programs comprising instructions, which when executed by one or more processors of a system, cause the system to: receive a selection of genomes for analysis as input, wherein the selection of genomes comprises a plurality of related genomes; identify clusters of orthologous genes (COGs) in the plurality of related genomes; determine a pairwise co-evolution metric, a pairwise co-regulation metric, a pairwise co-occurrence metric, a pairwise co-localization metric, or any combination thereof, for the identified COGs; determine pairwise functional association scores for the identified COGs based on the determined pairwise co-evolution metrics, pairwise co-regulation metrics, pairwise co-occurrence metrics, pairwise co-localization metrics, or any combination thereof; cluster the identified COGs according to their pairwise functional association scores to group functionally-related COGs; and output, based on a functional enrichment analysis performed on at least one COG cluster to identify COG clusters that are enriched for genes in a specific functional category, a determination that a COG cluster is a network of functionally-related genes in the specific functional category.

In some embodiments, the functional enrichment analysis does not require identification of a gene known to be associated with gene networks of a specific functional category. In some embodiments, the functional enrichment analysis comprises testing for enrichment of genes in functional categories known to be associated with biosynthetic gene clusters (BGCs), thereby identifying those COG clusters as putative BGCs. In some embodiments, the functional categories known to be associated with BGCs comprise gene ontology terms or KEGG pathways known to be associated with BGCs. In some embodiments, the functional enrichment analysis comprises testing for enrichment of protein domain representations known to be associated with biosynthetic gene clusters (BGCs), thereby identifying those COG clusters as putative BGCs. In some embodiments, the protein domain representations known to be associated with BGCs comprise PFAM domain representations, Conserved Domain Database (CDD) domain representations, or TIGRFAM domain representations known to be associated with BGCs.

In some embodiments, the non-transitory computer-readable medium further comprises instructions for identifying putative targets for a secondary metabolite synthesized by a putative BGC by: identifying protein sequences that are not components of known BGCs; determining pairwise functional association scores for the identified protein sequences and the putative BGC; and identifying putative targets for the secondary metabolite based on a comparison of the pairwise functional association scores to a first predetermined threshold. In some embodiments, the pairwise functional associate score comprises a co-regulation score, and the first predetermined threshold for the co-regulation score corresponds to a p-value of less than or equal to 0.05. In some embodiments, the pairwise functional association score comprises a co-evolution score, and the first predetermined threshold for the co-evolution score corresponds to a co-evolution score value of greater than or equal to 0.7. In some embodiments, the pairwise functional association score comprises a co-occurrence score, and the first predetermined threshold for the co-occurrence score corresponds to a co-occurrence score of greater than or equal to 0.5.

In some embodiments, identification of a putative BGC does not require identification of an associated core synthase.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### BRIEF DESCRIPTION OF THE DRAWINGS

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawings, of which:

FIG. 1 provides a non-limiting example of a process flowchart for identifying networks of functionally-related genes in accordance with one or more examples of the disclosure.

FIG. 2 provides a non-limiting schematic illustration of a computing device in accordance with one or more examples of the disclosure.

FIG. 3 provides a non-limiting schematic illustration of a pipeline for BGC discovery and functional assignment.

FIG. 4 provides a non-limiting example of performance metrics for a BGC identification pipeline for known and predicted BGCs. The figure shows the recall (fraction of genes known to be involved in the biosynthesis of the target molecule that was identified), precision (fraction of predicted genes that are true BGC genes for target molecule) and recall (core synthase clusters) (fraction of core synthase clusters predicted by antiSMASH to identified).

FIG. 5 provides a non-limiting example of data for the contribution of different components of a BGC identification pipeline to predictive performance. The figure shows the performance of the BGC identification pipeline when co-localization (Coloc only), co-localization and co-regulation (Coloc+Coreg), co-localization and co-evolution (Coloc+Coevo), or all of co-localization, co-regulation and co-evolution are applied (Coloc+Coreg+Coevo) for BGC prediction. Recall and precision are described elsewhere herein.

### DETAILED DESCRIPTION

A computational approach for identification of networks of functionally-related genes is described. In some instances, the computational approach may be used to identify, e.g., BGCs independently of an association with a core synthase, and may also facilitate linking BGCs to their potential downstream targets. The approach integrates information from co-evolution, co-occurrence, co-regulation, co-localization, and functional enrichment to: (i) group functionally-related genes across a gene network, (ii) delineate a specific type of gene network (e.g., a BGC), and (iii) propose targets for the associated secondary metabolites in the case that the methods are used to identify BGCs. The disclosed methods (and systems designed to implement the disclosed methods) are based on the observation that functionally related genes co-evolve (Steenwyk, et al. (2022), ibid.) and are often co-regulated. Furthermore, as previously stated, genes within gene networks such as BGCs are often co-localized and co-occur in related organisms. Details about the individual components of this approach and how to combine them into a pipeline for, e.g., BGC discovery and functional assignment are described below.

In some instances, the disclosed methods (e.g., computer-implemented methods) for identification of networks of functionally-related genes may comprise: receiving a selection of genomes for analysis as input, wherein the selection of genomes comprises a plurality of related genomes; identifying clusters of orthologous genes (COGs) in the plurality of related genomes; determining a pairwise co-evolution metric, a pairwise co-regulation metric, a pairwise co-occurrence metric, a pairwise co-localization metric, or any combination thereof, for the identified COGs; determining pairwise functional association scores for the identified COGs based on the determined pairwise co-evolution metrics, pairwise co-regulation metrics, pairwise co-occurrence metrics, pairwise co-localization metrics, or any combination thereof; clustering the identified COGs according to their pairwise functional association scores to group functionally-related COGs; and outputting, based on a functional enrichment analysis performed on at least one COG cluster to identify COG clusters that are enriched for genes in a specific functional category, a determination that a COG cluster is a network of functionally-related genes in the specific functional category.

In some instances the disclosed methods for identification of putative biosynthetic gene clusters (BGCs) may comprise: receiving a selection of genomes for analysis as input, where the selection of genomes comprises a plurality of related genomes; identifying clusters of orthologous genes (COGs) in the plurality of related genomes; determining a pairwise co-evolution metric, a pairwise co-regulation metric, a pairwise co-occurrence metric, a pairwise co-localization metric, or any combination thereof, for the identified COGs; determining pairwise functional association scores for the identified COGs based on the determined pairwise co-evolution metrics, pairwise co-regulation metrics, pairwise co-occurrence metrics, pairwise co-localization metrics, or any combination thereof; clustering the identified COGs according to their pairwise functional association scores to group functionally-related COGs; and outputting, based on a functional enrichment analysis performed on at least one COG cluster to identify COG clusters that are enriched for genes in a specific functional category and/or protein domains known to be associated with BGCs, a determination that a COG cluster is a putative BGCs.

In some instances, the disclosed methods further comprise identifying putative targets for a secondary metabolite synthesized by a putative BGC by: identifying protein sequences that are not components of known BGCs; determining pairwise functional association scores for the identified protein sequences and the putative BGC; and identifying putative targets for the secondary metabolite based on a comparison of the pairwise functional association scores to a first predetermined threshold.

### Definitions

Unless otherwise defined, all of the technical terms used herein have the same meaning as commonly understood by one of ordinary skill in the art in the field to which this disclosure belongs.

As used in this specification and the appended claims, the singular forms “a”, “an”, and “the” include plural references unless the context clearly indicates otherwise. Any reference to “or” herein is intended to encompass “and/or” unless otherwise stated, and encompasses any and all possible combinations of one or more of the associated listed items.

As used herein, the terms “includes, “including,” “comprises,” and/or “comprising” specify the presence of stated features, integers, steps, operations, elements, components, and/or units but do not preclude the presence or addition of one or more other features, integers, steps, operations, elements, components, units, and/or groups thereof.

As used herein, the term “about” a number refers to that number plus or minus 10% of that number. The term ‘about’ when used in the context of a range refers to that range minus 10% of its lowest value and plus 10% of its greatest value.

As used herein, a “secondary metabolite” refers to an organic small molecule or compound produced by archaea, bacteria, fungi or plants, which are not directly involved in the normal growth, development, or reproduction of the host organism, but are required for interaction of the host organism with its environment. Secondary metabolites are also known as natural products or genetically encoded small molecules. The term “secondary metabolite” is used interchangeably herein with “biosynthetic product” when referring to the product of a biosynthetic gene cluster.

The terms “biosynthetic gene cluster” or “BGC” are used herein interchangeably to refer to a locally clustered group of one or more genes that together encode a biosynthetic pathway for the production of a secondary metabolite. Exemplary BGCs include, but are not limited to, biosynthetic gene clusters for the synthesis of non-ribosomal peptide synthetases (NRPS), polyketide synthases (PKS), terpenes, and bacteriocins. See, for example, Keller N, “Fungal secondary metabolism: regulation, function and drug discovery.” Nature Reviews Microbiology 17.3 (2019): 167-180 and Fischbach M. and Voigt C. A., “Prokaryotic Gene Clusters: A Rich Toolbox For Synthetic Biology”, in: Institute of Medicine (US) Forum on Microbial Threats. The Science and Applications of Synthetic and Systems Biology: Workshop Summary. Washington (DC): National Academies Press (US); 2011. A21. BGCs contain genes encoding signature biosynthetic proteins that are characteristic of each type of BGC. The longest biosynthetic gene in a BGC is referred to herein as the “core synthase gene” of a BGC. In addition to genes involved in the biosynthesis of a secondary metabolite, a BGC may also include other genes, e.g., genes that encode products that are not involved in the biosynthesis of a secondary metabolite, which are interspersed among the biosynthetic genes. These genes are referred herein as being “associated” with the BGC if their products are functionally related to the secondary metabolite of the BGC. Some genes, e.g., genes not involved in the biosynthesis of a secondary metabolite produced by a BGC, are referred to herein as being “embedded” in the BGC if their products are functionally related to the secondary metabolite of the BGC and they are physically located in close proximity to the biosynthetic genes of the cluster. Some genes, e.g., genes not involved in the biosynthesis of a secondary metabolite produced by a BGC, are referred to herein as “non-embedded” if their products are functionally related to the secondary metabolite of a BGC but they are not physically located in close proximity to the biosynthetic genes of the BGC. An “anchor gene” refers to a biosynthetic gene (e.g., a core synthase) that is involved in the biosynthesis of a secondary metabolite produced by a BGC that is co-localized with a BGC and is known to be functionally related (i.e., associated) with the BGC.

The term “co-localize” refers to the presence of two or more genes in close spatial proximity, such as no more than about 200 kb apart, no more than about 100 kb apart, no more than about 50 kb apart, no more than about 40 kb apart, no more than about 30 kb apart, no more than about 20 kb apart, no more than about 10 kb apart, no more than about 5 kb apart, or less, in a genome.

The term “homolog” refers to a gene that is part of a group of genes that are related by descent from a common ancestor (i.e., gene sequences (i.e., nucleic acid sequences) of the group of genes and/or the sequences of their protein products are inherited through a common origin). Homologs may arise through speciation events (giving rise to “orthologs”), through gene duplication events, or through horizontal gene transfer events. Homologs may be identified by phylogenetic methods, through identification of common functional domains in the aligned nucleic acid or protein sequences, or through sequence comparisons.

The term “ortholog” refers a gene that is part of a group of genes that are predicted to have evolved from a common ancestral gene by speciation.

The terms “bidirectional best hit” and “BBH” are used herein interchangeably to refer to the relationship between a pair of genes in two genomes (i.e., a first gene in a first genome and a second gene is a second genome) wherein the first gene or its protein product has been identified as having the most similar sequence in the first genome as compared to the second gene or its protein product in the second genome, and wherein the second gene or its protein product has been identified as having the most similar sequence in the second genome as compared to the first gene or its protein product in the first genome. The first gene is the bidirectional best hit (BBH) of the second gene, and the second gene is the bidirectional best hit (BBH) of the first gene. BBH is a commonly used method to infer orthology.

As used herein, “sequence similarity” between two genes means similarity of either the nucleic acid (e.g., DNA, mRNA) sequences encoded by the genes or the amino acid sequences of the gene products.

“Percent (%) sequence identity” or “percent (%) sequence homology” with respect to the nucleic acid sequences (or protein sequences) described herein is defined as the percentage of nucleotide residues (or amino acid residues) in a candidate sequence that are identical or homologous with the nucleotide residues (or amino acid residues) in the oligonucleotide (or polypeptide) with which the candidate sequence is being compared, after aligning the sequences and considering any conservative substitutions as part of the sequence identity. Homology between different amino acid residues in a polypeptide is determined based on a substitution matrix, such as the BLOSUM (BLOcks SUbstitution Matrix) matrix. Methods for aligning sequences and determining percent sequence identity or percent sequence homology for nucleic acid or protein sequences are well known to those of skill in the art. Examples of publicly available computer software that may be used include, but are not limited to, BLAST (Basic Local Alignment Search Tool; software for comparing the amino-acid sequences of proteins or the nucleotide sequences of DNA and/or RNA molecules), BLAST-2, ALIGN or Megalign (DNASTAR) software. Any of a variety of suitable parameters for measuring sequence alignment and determining percent sequence identity or homology may be determined by those of skill in the art, including use of algorithms required to achieve maximal alignment over the full length of the sequences being compared.

Certain aspects of the present disclosure include process steps and instructions described herein in the form of an algorithm. It should be noted that the process steps and instructions of the present disclosure may be embodied in software, firmware, and/or hardware, and when embodied in software, may be downloaded to reside on and be operated from different platforms used by a variety of operating systems. Unless specifically stated otherwise in the following disclosure, it will be appreciated that descriptions utilizing terms such as “processing,” “computing,” “calculating,” “determining,” “displaying,” “generating” or the like, refer to the action and processes of a computer system, or similar electronic computing device, that manipulates and transforms data represented as physical (electronic) quantities within the computer system memories or registers or other such information storage, transmission, or display devices.

The section headings used herein are for organizational purposes only and are not to be construed as limiting the subject matter described. The description is presented to enable one of ordinary skill in the art to make and use the invention, and is provided in the context of a patent application and its requirements.

### Computational Methods to Identify Gene Networks Containing Functionally-Related Genes

Co-evolution of functionally associated genes: Genes involved in the same biological processes or pathways are generally faced with similar evolutionary pressures, and are thus likely to evolve at similar rates. Previous work analyzing the global co-evolution of genes in budding yeast indicated that functionally related genes show highly correlated rates of evolution, and that this property is not necessarily tied to the physical location of genes in the genome (see, e.g., Steenwyk, et al. (2022), ibid.). Networks derived from clustering co-evolving genes exhibited a high degree of functional coherence, with many essential genes involved in key biological process grouped together. This association of co-evolving genes mirrored observations from gene interaction networks derived from studying the impact on growth of single and double gene knockout mutants (Costanzo, et al. (2016), “A Global Genetic Interaction Network Maps a Wiring Diagram of Cellular Function”, Science 353(6306):aaf1420). Thus, co-evolution can be used as an approach to group functionally associated genes.

The disclosed methodology for evaluating co-evolving genes may comprise the following:
i. A diverse but related set of genomes from, e.g., fungal, bacterial, or plant species, is selected to facilitate capture of bona fide evolutionary signals.
ii. Orthologs shared between these species are then identified via bidirectional best BLAST hit and/or approaches using software tools such as orthoMCL and orthoFinder (see, e.g., Lee, et al. (2003), “OrthoMCL: Identification of Ortholog Groups for Eukaryotic Genomes”, Genome Res. 13(9):2178-89; Emms, et al. (2019), “OrthoFinder: Phylogenetic Orthology Inference for Comparative Genomics”, Genome Biol. 20(1):238) and grouping them into clusters of orthologous genes (COGs).
iii. To estimate the rate of co-evolution between the COGs, the percent identities between all pairs of proteins within each COG are computed. For each COG, the Pearson's correlation coefficient is then computed between it and every other COG identified that has a minimum number of shared pairs (i.e., equivalent genome pairs), generating a correlation matrix. This provides an estimate on how similar the rates of evolution are between every pair of COGs.
iv. To generate a network of co-evolving COGs, Markov clustering (MCL) or hierarchical clustering can be used to group COGs showing the highest degree of co-evolution. To ensure that only strong co-evolutionary links are considered, Pearson's correlation coefficient below a specified threshold (for instance 0.75) can be excluded. This approach groups COGs into clusters of co-evolving genes.
v. Functional enrichment analysis (e.g., a statistical analysis to identify functional categories of genes (e.g., based on Gene Ontology terms) that are over-represented within a network of co-evolving COGs, where the functions are inferred from the set of annotated genomes selected for the analysis) performed on the network may then reveal a high degree of functional coherence within the clusters, with essential functions grouped into the primary cluster, while genes encoding auxiliary functions are grouped into smaller clusters.
vi. In some instances, clusters of COGs enriched for essential functions may be filtered from downstream analysis, as these COGs are unlikely to be involved in secondary metabolite biosynthesis.

Co-regulation of functionally associated genes: As functionally-related genes are often co-regulated, this can serve as an additional layer of information in the analysis. This can be achieved by identifying signatures of shared regulation (i.e., shared putative cis-regulatory elements or transcription factor binding sites (TFBS)) within and between COGs, as these binding sites are often conserved among related species.

The disclosed methodology for identifying co-regulated genes may comprise the following:
i. To identify co-regulated genes, first the intergenic regions of genes within each COG (identified as described above) are extracted.
ii. De novo motif detection can be conducted on these intergenic regions using motif detection software such as MEME (see, e.g., Bailey, et al. (2015), “The MEME Suite”, Nucleic Acids Res. 43(W1):W39-49) or HOMER (see, e.g., Heinz, et al. (2010), “Simple Combinations of Lineage-Determining Transcription Factors Prime Cis-Regulatory Elements Required for Macrophage and B Cell Identities”, Mol Cell. 38(4): 576-589.).
iii. Putative TFBSs identified for each COG via this analysis can then be compared to TFBSs identified across all other COGs to generate a pairwise similarity matrix between COGs based on similarity of their identified motifs.
iv. To identify a network of co-regulated COGs, these pairwise motif similarity scores may then be used to cluster the COGs (e.g., via MCL or hierarchical clustering) to generate co-regulated COG clusters. To ensure that only highly significant motifs similarities are considered when clustering co-regulated COGs, a p-value cutoff (for instance, p-values of greater than or equal to 0.01) can be utilized to filter COGs by motif similarity scores.
v. Functional enrichment analysis performed on the gene network can be used to identify COG clusters for which a high degree of functional coherence exists.

Co-occurrence of COGs: As genes in a gene network (e.g., a BGC) need to function together (e.g., to produce a target secondary metabolite), they can be expected to show a correlated pattern in their occurrence across genomes, with all genomes capable of producing, e.g., a secondary metabolite, sharing a core set of genes.

The disclosed methodology for evaluating co-occurring genes may comprise the following:
i. To utilize co-occurrence in identifying gene networks, a co-occurrence score can be calculated for each pair of COGs by computing the Jaccard index of the set of genomes in each COG. For instance, to generate a co-occurrence score between COG A and COB A, the Jaccard index is computed:

Co-occurrence score=(|A∩B|)/(|A∪B|)
where A∩B is the number of genomes shared between COG A and COG B, and A∪B is the set of all genomes present in A or B.
ii. This provides a measure of the pairwise co-occurrence between COGs ranging from 0 (for COGs that occur in completely different genomes) to 1 (for COGs occurring in the exact same genomes). A pairwise co-occurrence matrix can thus be computed between all COGs.

Co-localization of COGs: Networks of functionally related genes (e.g., BGCs) are often made up of genes located in close proximity to each other within the genome. This information can also be integrated to facilitate grouping of functionally related genes.

The disclosed methodology for incorporating information about co-localization may comprise the following:
i. To leverage information about co-localization, a proximity score can be generated that captures how close orthologous genes are positioned with respect to one another across species. A non-limiting example of such a proximity score could be given by:

$$Proximity score = 1 / ( 1 + \# of interspacing genes )$$

where adjacent genes would receive a score of 1, more distant genes would receive a score less than 1 (the more distant the genes the smaller the score) and genes on separate contigs would receive a value of 0.
ii. Thus, in comparing two COGs the proximity score for each gene in COG A to its counterpart gene from the same genome in COG B can be calculated and an average for all computable pairs taken to obtain an average proximity score for COG A to COG B. A pairwise co-localization matrix can thus be computed between all COGs.
iii. As this measure of colocalization is computed across species, it also captures synteny between genes. This can thus be used to cluster COGs, grouping syntenous COGs together.

Signatures of horizontal gene transfer: BGCs can be exchanged between microbial species via the process of horizontal gene transfer, thereby driving increased diversity of natural products. As organisms have unique genomic properties, in some instances these laterally transferred genes may still possess genomic signatures of the original donor, particularly if they are recently acquired, or derived from very distantly related organisms, or the process of amelioration is slow. Thus, genomic properties such as codon utilization, GC content and/or dinucleotide ratio, may differ significantly between, e.g., horizontally transferred BGCs, and the rest of the host genome, thereby providing another metric that may be used to delineate gene networks such as BGCs.

The codon adaptation index (CAI) of a gene sequence, for example, can be computed relative to the rest of the genome (which serves as the reference) by first computing the relative synonymous codon utilization (RSCU) across the genome as previously described (see, e.g., Sharp, et al. (1987), “The Codon Adaptation Index—A Measure of Directional Synonymous Codon Usage Bias, and Its Potential Applications”, Nucleic Acids Res. 15(3):1281-95). By comparing the frequency of codon utilization of a gene to the RSCU table, CAI is computed. CAI is a value between 0 and 1 that serves as a measure of how similar the codon usage of a gene or locus is to a reference set. Genes with higher CIA values have a more similar codon usage in comparison to the rest of the genome. The CIA can be computed for all genes in the genome, and those with lower-than-expected values can be considered candidate horizontally transferred genes.

Co-localized, co-occurring, co-regulated and/or co-evolving gene clusters may then be further refined by clustering based on CAI. Alternatively, CAI can be employed as part of post-processing (described below), to retrieve nearby horizontally transferred genes missed in upstream clustering steps.

Dinucleotide relative abundance (DRA) is the ratio of the observed dinucleotide frequencies to the expected frequencies derived from single nucleotide frequencies under an independence assumption, and may be computed using the formula:

$${p}_{xy} = \frac{{f}_{xy}}{{f}_{x} {f}_{y}}$$

where p_{xy} is the DRA for dinucleotide xy, f_{xy} is the dinucleotide frequency, and fₓ, f_{y} are the single nucleotide frequencies. DRA values computed for target genomic loci, for instance an entire BGC or a specific gene, can be compared to overall/background DRA values derived from the whole genome to generate a dinucleotide signature dissimilarity index (DSDI) by taking, e.g., the Euclidean distance, between the two vectors. In some instances, the DSDI may be generated by taking the chi-squared/DRA-divergence, delta-distance, or quadratic discriminant between the two vectors (see, e.g., Baran, et al. (2008), “Detecting Horizontally Transferred and Essential Genes Based on Dinucleotide Relative Abundance”, DNA Research 15:267-276). The DSDI can be computed for all genes in the genome, and those with higher-than-expected values can be considered candidate horizontally transferred genes.

Co-localized, co-occurring, co-regulated and/or co-evolving COGs may then be further refined by clustering based on DSDI. Alternatively, DSDI can be employed as part of post-processing (described below), to retrieve nearby horizontally transferred genes missed in upstream clustering steps. In some instances, CAI and DSDI may be combined for this analysis.

Clustering functionally-related genes: To cluster functionally related genes, one can then combine these four computed measures for pairwise co-evolution (Pearson's correlation coefficient), pairwise co-regulation (motif similarity score), pairwise co-localization (average proximity score) and pairwise co-occurrence (co-occurrence score), or a combination or subset thereof, into a unified score for functional association. For example, in some instances, a unified score for functional association may be computed based on an additive score of any two or more of the pairwise metrics for co-evolution, co-regulation, co-localization, co-occurrence, and optionally including horizontal gene transfer, where the individual metrics may be weighted equally in some instances, or may be weighted differently in some instances according to, e.g., a rank-ordering of their predictive value. This pairwise functional association score can then be used to cluster the COGs, e.g., using either MCL or hierarchical clustering, to group functionally-associated COGs.

Functional enrichment analysis to identify potential gene networks: Following the methods outlined above results in a grouping of all functionally related COGs into clusters, however, to identify functional gene networks, e.g., biosynthetic gene clusters, among the identified COG clusters that are likely involved in, e.g., the synthesis of secondary metabolites, functional enrichment analysis can be conducted to test for enrichment of specified functional categories (using, e.g., Gene Ontology terms and/or KEGG pathways) and/or specified protein domains (e.g., PFAM domains) known to be associated with, e.g., BGCs, such as methyl-transferases, mono-oxygenases, etc. COG clusters enriched for the specified functional categories and/or protein domains can then be earmarked as putative gene networks, e.g., putative BGCs.

Post-processing: To ensure that all components of a gene network such as a BGC are captured, post-processing steps can be performed to capture proximal genes that might be unique to specific clades or species. Postprocessing steps may include, but are not limited to, conversion of COG-level information (across species) to genome-level information for each genome selected for the analysis. For each genome, each identified cluster of functionally related genes can be augmented with embedded genes found between the cluster genes in specific genomes, but not found at COG-level. Similarly, the cluster can also be augmented with proximal genes having DNA signatures of horizontal gene transfer similar to other genes in the identified cluster of functionally related genes. Conversely, genes that are found to be very distant from the core set of functionally related genes in specific genomes maybe considered for exclusion from the cluster.

Downstream target association: To associate potential targets of a secondary metabolite to an identified BGC, proteins that are not typically BGC components and have high functional association scores can be easily identified (with or without considering of co-localization) to rank potential targets of the secondary metabolite.

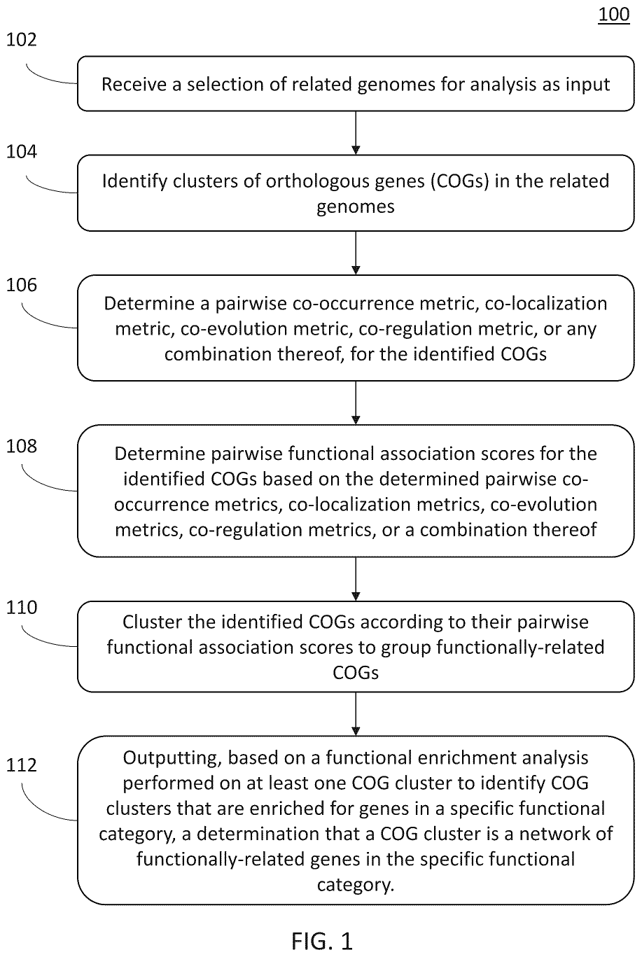

FIG. 1 provides a non-limiting example of a flowchart for a process 100 for identifying networks of functionally-related genes. Process 100 can be performed, for example, as a computer-implemented method using software running on one or more processors of one or more electronic devices, computers, or computing platforms. In some examples, process 100 is performed using a client-server system, and the blocks of process 100 are divided up in any manner between the server and a client device. In other examples, the blocks of process 100 are divided up between the server and multiple client devices. Thus, while portions of process 100 are described herein as being performed by particular devices of a client-server system, it will be appreciated that process 100 is not so limited. In other examples, process 100 is performed using only a client device or only multiple client devices. In process 100, some blocks are, optionally, combined, the order of some blocks is, optionally, changed, and some blocks are, optionally, omitted. In some examples, additional steps may be performed in combination with the process 100. Accordingly, the operations as illustrated (and described in greater detail below) are exemplary by nature and, as such, should not be viewed as limiting.

At step 102 in FIG. 1, a selection of genomes for analysis is received as input, where the selection of genomes comprises a plurality of related genomes. In some instances, for example, the selection of genomes may be input by a user of a system configured to perform the methods (e.g., computer-implemented methods) described herein.

In some instances, the plurality of related genomes may comprise genomes for organisms that are known to comprise, or are suspected of comprising, gene networks (e.g., networks of functionally-related genes or gene products) of interest. Examples of gene networks include, but are not limited to, biochemical, cellular, and signal transduction pathways such as gene regulatory networks, primary and secondary metabolic pathways, hormone signaling pathways, and the JAK-STAT pathway involved in immune response. In some instances, the plurality of related genomes may comprise, e.g., mammalian genomes, human genomes, avian genomes, reptilian genomes, amphibian genomes, plant genomes, fungal genomes, bacterial genomes, or viral genomes.

In some instances, the gene networks of interest may comprise BGCs that produce secondary metabolites, and the plurality of related genomes may comprise, e.g., fungal genomes, bacterial genomes, or plant genomes.

In some instances, the plurality of related genomes may be input in any of a variety of formats or representations known to those of skill in the art. Examples include, but are not limited to, nucleotide sequences, amino acid sequences, or sequences of CDD, Gene3D, PANTHER, Pfam, ProSitePatterns, ProSiteProfiles, SUPERFAMILY, SMART, TIGRFAM, SFLD, Hamap, Coils, PRINTS, PIRSR, AntiFam, MobiDBLite, or PIRSF representations, or any combination thereof, of protein domains encoded by genes within a genome of the plurality of related genomes. In some instances, the sequence of protein domain representations for a genome is generated by a process comprising retrieving a protein sequence for each gene in the genome and identifying protein domains in a protein sequence by sequence alignment against a database of, for example, CDD, Gene3D, PANTHER, Pfam, ProSitePatterns, ProSiteProfiles, SUPERFAMILY, SMART, TIGRFAM, SFLD, Hamap, Coils, PRINTS, PIRSR, AntiFam, MobiDBLite, or PIRSF representations, respectively.

In some instances, the representation of a genome may further comprise associated gene ontology (GO) terms, an identification of any known resistance genes that are present in the genome, an identification of additional regulatory elements such as promoters, enhancers, or silencers, that are present in the genome, or an identification of additional epigenetic elements, such as histone folding, DNA methylation or acetylation, that are present in the genome.

In some instances, the plurality of related genomes received as input may comprise 2, 3, 4, 5, 6, 7, 8, 9, 10, 20, 30, 40, 50, 60, 70, 80, 90, 100, or more than 100 genomes, or any number of genomes within this range.

At step 104 in FIG. 1, clusters of orthologous genes (COGs) may be identified in the plurality of related genomes, as described elsewhere herein. In some instances, for example, the identification of COGs may comprise identifying orthologous genes in the plurality of related genomes as bidirectional best hits (BBHs) using BLAST, followed by clustering of the identified orthologous genes. In some instances, identifying COGs may comprise identifying orthologous genes in the plurality of related genomes using a software tool such as orthoMCL (Li, et al. (2003), “OrthoMCL: Identification of Ortholog Groups for Eukaryotic Genomes”, Genome Research 13:2178-2189) or orthoFinder (Emms, et al. (2015), “OrthoFinder: Solving Fundamental Biases in Whole Genome Comparisons Dramatically Improves Orthogroup Inference Accuracy”, Genome Biology 16:157).

At step 106 in FIG. 1, a pairwise co-occurrence metric, pairwise co-localization metric, pairwise co-evolution metric, pairwise co-regulation metric, or any combination thereof, may be determined for the identified COGs, as described elsewhere herein.

In some instances, for example, determining a pairwise co-evolution metric for COGs may comprise: computing a percent identity between each pair of protein sequences within each COG of a pair of COGs to identify shared protein sequences; computing a Pearson's correlation coefficient for each pair of COGs that include a specified minimum number of shared protein sequences to estimate a rate of co-evolution; filtering the COGs by excluding COGs for which the pairwise Pearson's correlation coefficient is less than a predetermined threshold and clustering the remaining COGs according to the estimated rates of co-evolution; and performing a functional enrichment analysis to exclude clusters of COGS that are enriched for essential metabolic functional categories.

In some instances, the specified minimum number of shared protein sequences used to identify COGs for which a Pearson's correlation coefficient may be calculated may be 5, 10, 20, 30, 40, 50, 60, 70, 80, 90, 100, or more than 100 shared protein sequences.

In some instances, the predetermined threshold for Pearson's correlation coefficient used to exclude COGs from clustering may correspond to a Pearson's correlation coefficient value of 0.7, 0.8, 0.9, 0.95, 0.98, or 0.99, or any value within this range.

In some instances, clustering the remaining COGs according to the estimated rates of co-evolution may comprise the use of a Markov clustering (MCL) or hierarchical clustering algorithm.

In some instances, determining a pairwise co-regulation metric for COGs may comprise: extracting intergenic regions within each COG; performing a de novo detection of sequence motifs within the extracted intergenic regions to identify putative cis-regulatory elements or transcription factor binding sites (TFBS); comparing the putative cis-regulatory elements or TFBS identified for each COG to those identified across all other COGs to determine pairwise motif similarity scores between COGs; filtering the COGs to exclude COGs for which pairwise motif similarity scores have a p-value of less than or equal to a predetermined threshold; and clustering the filtered COGs based on the pairwise motif similarity scores to identify co-regulated COG clusters.

In some instances, the predetermined threshold for pairwise motif similarity score p-values may correspond to a p-value of 0.001, 0.01, 0.02, 0.03, 0.04, 0.05, or any p-value within this range.

In some instances, clustering the remaining COGs according to the motif similarity scores may comprise the use of a Markov clustering (MCL) or hierarchical clustering algorithm.

In some instances, determining a pairwise co-occurrence metric for COGs may comprise: computing a Jaccard index for each pair of COGs (COG A and COG B) based on a relationship:

Co-occurrence score=(|A∩B|)/(|A∪B|)

filtering the COGs to exclude COGs for which pairwise co-occurrence scores have a value of less than a predetermined threshold; and clustering the filtered COGs based on the pairwise co-occurrence scores to identify co-occurring COG clusters.

In some instances, the predetermined threshold for pairwise co-occurrence scores may correspond to a pairwise co-occurrence score value of 0.3, 0.4, 0.5, 0.6, 0.7, 0.8, or 0.9, or any value within this range.

In some instances, clustering the remaining COGs according to the co-occurrence scores may comprise the use of a Markov clustering (MCL) or hierarchical clustering algorithm.

In some instances, determining a pairwise co-localization metric for COGs may comprise: computing a proximity score for each pair of corresponding gene sequences in a pair of COGS (COG A and COG B) based on a relationship:

$$Proximity score = 1 / ( 1 + \# of interspacing genes$$

averaging the proximity scores to determine the co-localization metric for each pair of COGs; and clustering the COGs based on the averaged proximity score (co-localization metric) to identify co-localized COG clusters.

In some instances, clustering of COGs according to the averaged proximity score may comprise the use of a Markov clustering (MCL) or hierarchical clustering algorithm. In some instances, the clustering is performed using a Markov clustering (MCL) algorithm and an MCL inflation parameter value ranging from 1.5 to 3.0. In some instances, the MCL inflation parameter value may be 1, 1.5, 2, 2.5, 3, 3.5, 4, 4.5, 5, or any value within this range of values.

At step 108 in FIG. 1, pairwise functional association scores for the identified COGs are determined based on the pairwise co-occurrence metrics, co-localization metrics, co-evolution metrics, co-regulation metrics, or a combination thereof, that were determined at step 106.

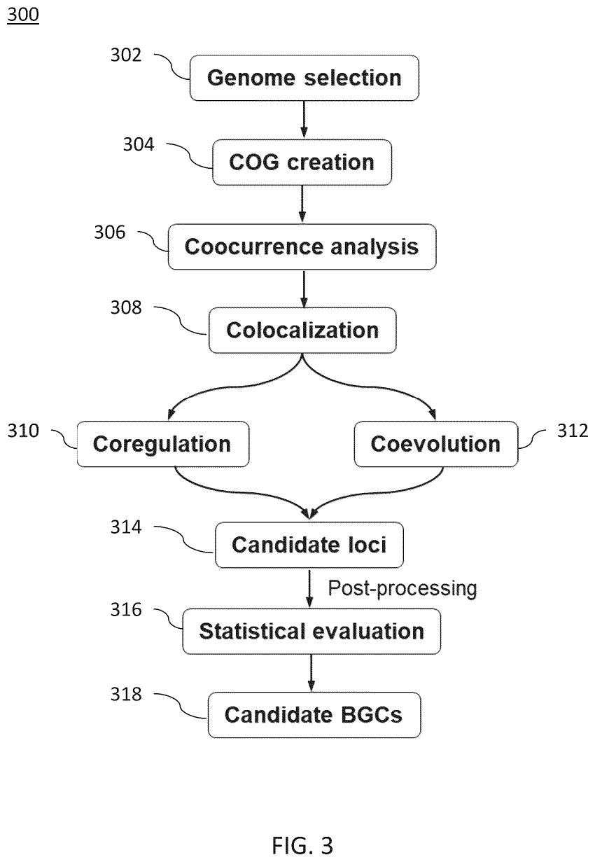

In some instances, the pairwise functional association score for the identified COGs comprises an algebraic function of the determined pairwise co-evolution metrics, pairwise co-regulation metrics, pairwise co-occurrence metrics, pairwise co-localization metrics, or any combination thereof. In some instances, for example, the pairwise functional association score for the identified COGs is based on addition of the determined pairwise co-evolution metrics, pairwise co-regulation metrics, pairwise co-occurrence metrics, pairwise co-localization metrics, or any combination thereof. Such a pairwise functional association score may have a value ranging from 0 to 1, with higher values indicating higher functional association. In some instances, a pairwise functional association score value of, e.g., greater than or equal to 0.5, 0.6, 0.7, or 0.8 might be used as a cutoff threshold to be applied prior to clustering. In some instances, as depicted in FIG. 3, the pairwise co-evolution metrics, pairwise co-regulation metrics, pairwise co-occurrence metrics, pairwise co-localization metrics may be applied sequentially as an alternative to combining them in a single pairwise functional associate score.

At step 110 in FIG. 1, the identified COGs are clustered according to their pairwise functional association scores to group functionally-related COGs. In some instances, clustering the identified COGs according to their pairwise functional association scores may comprise the use of a Markov clustering (MCL) or hierarchical clustering algorithm.

In some instances, the method may further comprise determining a horizontal gene transfer metric based on computing, for example, a codon adaptation index (CAI) or a dinucleotide signature dissimilarity index (DSDI). CAI values, for example, may range from 0 to 1 (e.g., values of 0, 0.1, 0.2, 0.3, 0.4, 0.5, 0.6, 0.7, 0.8, 0.9, 0.95, 1.0, or any value within this range), with lower values being indicative of horizontal gene transfer. In some instances, the horizontal gene transfer metric may be used to further refine clustering of co-localized, co-occurring and/or co-evolving COGs.

In some instances, the horizontal gene transfer metric may be used as part of a post-processing step to retrieve nearby horizontally transferred genes missed in upstream clustering steps.

At step 112 in FIG. 1, a determination that a COG cluster is a network of functionally-related genes in the specific functional category is output based on a functional enrichment analysis performed on at least one COG cluster to identify COG clusters that are enriched for genes in a specific functional category. In some instances, identification of a putative gene network does not require identification of an associated gene known to be associated with gene networks of a given functional category.

In some instances, the functional enrichment analysis may comprise testing for enrichment of genes in functional categories known to be associated with, e.g., biosynthetic gene clusters (BGCs), to thereby identify those COG clusters as putative BGCs. The functional categories known to be associated with BGCs may comprise, for example, gene ontology terms or KEGG pathways known to be associated with BGCs.

Examples of gene ontology terms known to be associated with BGCs include, but are not limited to, GO:0019748 (secondary metabolic process), GO:0044550 (secondary metabolite biosynthetic process), GO:0030639 (polyketide biosynthetic process), GO:0030638 (polyketide metabolic process), GO:0043455 (regulation of secondary metabolic process), GO:1900539 (fumonisin metabolic process) or any combination thereof.

Examples of KEGG pathways known to be associated with BGCs include, but are not limited to, M00778 (Type II polyketide backbone biosynthesis), M00095 (C5 isoprenoid biosynthesis, mevalonate pathway), M00937 (Aflatoxin biosynthesis), M00893 (Lovastatin biosynthesis), or any combination thereof.

In some instances, the functional enrichment analysis may comprise testing for enrichment of protein domain representations known to be associated with, e.g., biosynthetic gene clusters (BGCs), thereby identifying those COG clusters as putative BGCs.

Examples of protein domain representations known to be associated with BGCs include, but are not limited to, PFAM domain representations, Conserved Domain Database (CDD) domain representations, or TIGRFAM domain representations known to be associated with BGCs. Examples of protein domains associated with BGCs include, but are not limited to, PF00550 (Phosphopantetheine attachment site), PF00501 (AMP-binding enzyme), PF07690 (Major Facilitator Superfamily), PF00067 (Cytochrome P450), PF00698 (Acyl transferase domain), PF08242 (Methyltransferase domain), or any combination thereof.

In some instances, the method may further comprise identifying putative targets for a secondary metabolite synthesized by a putative BGC by: identifying protein sequences that are not components of known BGCs; determining pairwise functional association scores for the identified protein sequences and the putative BGC; and identifying putative targets for the secondary metabolite based on a comparison of the pairwise functional association scores to a predetermined threshold. In some instances, identification of a putative BGC does not require identification of an associated core synthase.

In some instances, the pairwise functional associate score may comprise a co-regulation score, and the predetermined threshold for the co-regulation score corresponds to a p-value of, for example, less than or equal to 0.05, 0.04, 0.03, 0.02, 0.01 or 0.001.

In some instances, the pairwise functional association score may comprise a co-evolution score, and the first predetermined threshold for the co-evolution score corresponds to a co-evolution score value of greater than or equal to 0.7, 0.8, 0.9, or 0.95.

In some instances, the pairwise functional association score may comprise a co-occurrence score, and the first predetermined threshold for the co-occurrence score corresponds to a co-occurrence score of greater than or equal to 0.5, 0.6, 0.7, 0.8, 0.9, or 0.95.

### Methods of Use

The disclosed computer-based methods for identifying gene networks, and in particular for identifying BGCs, have various applications including, for example, performing further evaluation of the genes predicted to be part of a BGC to: (i) identify homologs or orthologs of one or more target sequences (e.g., gene sequences) of interest in one or more target genomes, (ii) identify a resistance gene against a secondary metabolite produced by a BGC in a target genome, (iii) predict a function of a secondary metabolite produced by a BGC, and/or (iv) identify a BGC that encodes biosynthetic enzymes for producing a secondary metabolite having an activity of interest (e.g., a therapeutic activity of interest), etc.

Methods for evaluating genes embedded within or associated with a BGC to identify resistance genes (e.g., “embedded target genes” (ETaGs) or “non-embedded target genes” (NETaGs)) have been described in International Patent Application Nos. PCT/US2022/049016, PCT/US2022/049040, PCT/US2022/079965, and PCT/US2022/080447, the contents of each of which are incorporated herein in their entirety. In some instances, for example, a method for identifying resistance genes (e.g., embedded target genes (ETaGs) and/or non-embedded target genes (NETaGs)) may comprise: receiving a selection of at least one target sequence of interest; receiving a selection of target genomes from a genomics database, wherein the selection of target genomes comprises a plurality of target genomes from organisms that are known to produce, or are likely to produce, secondary metabolites; performing a search to identify homologs of the at least one target sequence in the plurality of target genomes; generating a phylogenetic tree based on the identified homologs of the at least one target sequence; classifying the genomes of the plurality of target genomes as positive genomes or negative genomes based on the phylogenetic tree, wherein positive genomes are genomes that belong to a clade for which multiple copies of the at least one target sequence homolog are present, wherein negative genomes are genomes that belong to a clade for which a single copy of the at least one target sequence homolog is present; and wherein a target sequence homolog that is present in multiple copies in a positive genome is a putative resistances genes (e.g., a pETaG or pNETaG); determining, based at least in part on the classification of positive and negative genomes, at least one genomic parameter selected from the following: i) one or more scores indicative of co-occurrence of the at least one target sequence homolog (putative resistance gene (e.g., a pETaG or pNETaG)) and one or more genes associated with a biosynthetic gene cluster (BGC); ii) one or more scores indicative of co-evolution of the at least one target sequence homolog (putative resistance gene (e.g., a pETaG or pNETaG)) and one or more genes associated with a BGC; iii) one or more scores indicative of co-regulation of the at least one target sequence homolog (putative resistance gene (e.g., a pETaG or pNETaG)) with one or more genes associated with a BGC; and iv) one or more scores indicative of co-expression of the at least one target sequence homolog (putative resistance gene (e.g., a pETaG or pNETaG)) with one or more genes associated with a BGC; and determining, based on the at least one genomic parameter, a likelihood that the putative resistance gene (e.g., pETaG or pNETaG) is a resistance gene (e.g., an embedded target gene (ETaG) or non-embedded target gene (NETaG)).

In some instances, determining the likelihood that the putative resistance gene is a resistance gene may comprise comparing the at least one determined genomic parameter to at least one predetermined threshold.

In some instances, the selection of at least one target sequence of interest may be provided as input by a user of a system configured to perform the computer-implemented method. In some instances, for example, the at least one target sequence of interest may comprise a sequence of a gene identified as belonging to a BGC by any of the methods described elsewhere herein.

In some instances, the at least one target sequence of interest may comprise an amino acid sequence, a nucleotide sequence, or any combination thereof. In some instances the at least one target sequence of interest may comprise a peptide sequence or portion thereof, a protein sequence or portion thereof, a protein domain sequence or portion thereof, a gene sequence or portion thereof, or any combination thereof. In some instances, the at least one target sequence of interest may comprise a mammalian sequence, a human sequence, a plant sequence, a fungal sequence, a bacterial sequence, an archaea sequence, a viral sequence, or any combination thereof.

In some instances, the at least one target sequence of interest may comprise a primary target sequence and one or more related sequences. In some instances, the one or more related sequences may comprise sequences that are functionally-related to the primary target sequence. In some instances, the one or more related sequences may comprise sequences that are pathway-related to the primary target sequence.

In some instances, the selection of target genomes may be provided as input by a user of a system configured to perform the computer-implemented method. In some instances, the plurality of target genomes may comprise plant genomes, fungal genomes, bacterial genomes, or any combination thereof. In some instances, the genomics database may comprise a public genomics database. In some instances, the genomics database comprises a proprietary genomics database.

In some instances, the search to identify homologs of the at least one target sequence (e.g., homologs of a gene sequence identified as belonging to a BGC) may comprise identification of homologs based on probabilistic sequence alignment models. In some instances, the probabilistic sequence alignment models are profile hidden Markov models (pHMMs). In some instances, homologs are identified based on a comparison of probabilistic sequence alignment model scores to a predefined threshold.

In some instances, the search to identify homologs of the at least one target sequence may comprise identification of homologs based on alignment of sequences using a local sequence alignment search tool, calculation of a sequence homology metrics based on the alignments, and comparison of the calculated sequence homology metrics to a predefined threshold. In some instances, the local sequence alignment search tool comprises BLAST, DIAMOND, HMMER, Exonerate, or ggsearch. In some instances, the predefined threshold may comprise a threshold for percent sequence identity, percent sequence coverage, E-value, or bitscore value.

In some instances, the search to identify homologs of the at least one target sequence may comprise identification of homologs based on use of a gene and/or protein domain annotation tool. In some instances, the gene and/or protein domain annotation tool comprises InterProScan or EggNOG.

In some instances, the generation of phylogenetic trees based on the identified homologs of the at least one target sequence may comprise alignment of homolog sequences using an alignment software tool, trimming of the aligned homolog sequences using a sequence trimming software tool, and construction of a phylogenetic tree using phylogenetic tree building software tool. In some instances, the alignment software tool comprises MAFFT, MUSCLE, or ClustalW. In some instances, the sequence trimming software tool comprises trimAI, GBlocks, or ClipKIT. In some instances, the phylogenetic tree building software tool comprises FastTree, IQ-TREE, RAxML, MEGA, MrBayes, BEAST, or PAUP. In some instances, the construction of the phylogenetic tree may be based on a maximum likelihood algorithm, parsimony algorithm, neighbor joining algorithm, distance matrix algorithm, or Bayesian inference algorithm.

In some instances, the one or more scores indicative of co-occurrence may be determined based on identifying positive correlations between the presence of multiple copies of a putative resistance gene and the presence of the one or more genes of a BGC in positive genomes. In some instances, identifying the positive correlations between the presence of multiple copies of the putative resistance gene and the presence of the one or more genes of a BGC in positive genomes may comprise the use of a clustering algorithm to cluster aligned protein sequences, aligned nucleotide sequences, aligned protein domain sequences, or aligned pHMMs for a group of BGCs to identify BGC communities within the plurality of target genomes. In some instances, identifying the positive correlations between the presence of multiple copies of the putative resistance gene and the presence of the one or more genes of a BGC in positive genomes may comprise the use of a phylogenetic analysis of protein sequences or protein domains for a group of BGCs to identify BGC communities within the plurality of target genomes. In some instances, identifying the positive correlations between the presence of multiple copies of the putative resistance gene and the presence of the one or more genes of a BGC in positive genomes may comprise choosing genomes with a specific taxonomy to identify BGC communities within the plurality of target genomes.

In some instances, the one or more scores indicative of co-evolution of a putative resistance gene and the one or more genes associated with a BGC may be determined based on a co-evolution correlation score, a co-evolution rank score, a co-evolution slope score, or any combination thereof. In some instances, the co-evolution correlation score may be based on a correlation between pairwise percent sequence identities of a cluster of orthologous groups (COG) for the putative resistance gene and pairwise percent sequence identities of a cluster of orthologous groups (COG) for one of the one or more genes associated with a BGC. In some instances, the co-evolution rank score may be based on a ranking of a correlation coefficient of a COG that contains one of the one or more genes associated with a BGC in ascending order in relation to a COG that contains the putative resistance gene. In some instances, in the case of ties for a distance score, the rank for all COGs in the tie may be set equal to a lowest rank in the group. In some instances, the co-evolution slope score may be based on an orthogonal regression of pairwise percent sequence identities of a COG for the putative resistance gene and pairwise percent sequence identities of a COG for one of the one or more genes associated with a BGC. In some instances, only COGs arising from unique positive genomes that have more than three genes remaining after removing corresponding genes from negative genomes are used to evaluate a co-evolution correlation score, a co-evolution rank score, or a co-evolution slope score.

In some instances, the one or more scores indicative of co-regulation may be based on DNA motif detection from intergenic sequences of the one or more genes associated with a BGC and the putative resistance gene.

In some instances, the one or more scores indicative of co-expression may be based on a differential expression analysis and/or a clustering analysis of global transcriptomics data.

In some instances, the one or more genes associated with a biosynthetic gene cluster (BGC) may comprise an anchor gene, a core synthase gene, a biosynthetic gene, a gene not involved in the biosynthesis of a secondary metabolite produced by the BGC, or any combination thereof.

In some instances, the putative resistance gene may be a putative embedded target gene (pETaG) or a putative non-embedded target gene (pNETaG).

In some instances, the resistance gene may be an embedded target gene (ETaG) or a non-embedded target gene (NETaG).

In some instances, a method for predicting a function of a secondary metabolite may comprise: receiving a selection of at least one target sequence of interest, wherein the at least one target sequence of interest corresponds to a gene sequence associated with a biosynthetic gene cluster (BGC) known to produce the secondary metabolite; receiving a selection of target genomes from a genomics database, wherein the selection of target genomes comprises a plurality of target genomes from organisms that are known to produce secondary metabolites; performing a search to identify homologs of the at least one target sequence in the plurality of target genomes; generating a phylogenetic tree based on the identified homologs of the at least one target sequence; classifying the genomes of the plurality of target genomes as positive genomes or negative genomes based on the phylogenetic tree, wherein positive genomes are genomes that belong to a clade for which multiple copies of the at least one target sequence homolog are present, wherein negative genomes are genomes that belong to a clade for which a single copy of the at least one target sequence homolog is present; and wherein a target sequence homolog that is present in multiple copies in a positive genome is a putative resistance gene; determining, based at least in part on the classification of positive and negative genomes, at least one genomic parameter selected from the following: i) one or more scores indicative of co-occurrence of the at least one target sequence homolog (putative resistance gene) and one or more genes associated with the BGC; ii) one or more scores indicative of co-evolution of the at least one target sequence homolog (putative resistance gene) and one or more genes associated with the BGC; iii) one or more scores indicative of co-regulation of the at least one target sequence homolog (putative resistance gene) with one or more genes associated with the BGC; and iv) one or more scores indicative of co-expression of the at least one target sequence homolog (putative resistance gene) with one or more genes associated with the BGC; and determining, based on the at least one genomic parameter, a likelihood that the putative resistance gene is a resistance gene that encodes a protein target that is acted upon by the secondary metabolite.

In some instances, a method for identifying a biosynthetic gene cluster (BGC) that encodes biosynthetic enzymes for producing a secondary metabolite having an activity of interest may comprise: receiving a selection of at least one target sequence of interest, wherein the at least one target sequence of interest comprises a sequence that encodes a therapeutic target of interest; receiving a selection of target genomes from a genomics database, wherein the selection of target genomes comprises a plurality of target genomes from organisms that are known to produce secondary metabolites; performing a search to identify homologs of the at least one target sequence in the plurality of target genomes; generating a phylogenetic tree based on the identified homologs of the at least one target sequence; classifying the genomes of the plurality of target genomes as positive genomes or negative genomes based on the phylogenetic tree, wherein positive genomes are genomes that belong to a clade for which multiple copies of the at least one target sequence homolog are present, wherein negative genomes are genomes that belong to a clade for which a single copy of the at least one target sequence homolog is present; and wherein a target sequence homolog that is present in multiple copies in a positive genome is a putative resistance gene; determining, based at least in part on the classification of positive and negative genomes, at least one genomic parameter selected from the following: i) one or more scores indicative of co-occurrence of the at least one target sequence homolog (putative resistance gene) and one or more genes associated with a biosynthetic gene cluster (BGC); ii) one or more scores indicative of co-evolution of the at least one target sequence homolog (putative resistance) and one or more genes associated with a BGC; iii) one or more scores indicative of co-regulation of the at least one target sequence homolog (putative resistance gene) with one or more genes associated with a BGC; and iv) one or more scores indicative of co-expression of the at least one target sequence homolog (putative resistance gene) with one or more genes associated with a BGC; and determining, based on the at least one genomic parameter, a likelihood that the putative resistance gene is an actual resistance gene associated with a BGC that produces a secondary metabolite that acts upon a protein product encoded by the resistance gene.

In some instances, the methods of the present disclosure may further comprise performing an in vitro assay, for example, an assay to detect or measure an activity (e.g., a receptor binding activity, an enzyme activation activity, an enzyme inhibition activity, etc.) of a secondary metabolite (or analog thereof) on a mammalian (e.g., human) protein encoded by a mammalian (e.g., human) gene that is homologous to an ETaG or NETaG identified in an organism comprising a biosynthetic gene cluster (BGC) that produces the secondary metabolite. In some instances, the methods may further comprise performing an in vitro assay to detect or measure an activity (e.g., a receptor binding activity, an enzyme activation activity, an enzyme inhibition activity, etc.) of a secondary metabolite (or analog thereof) on a protein (e.g., a reptilian, avian, amphibian, plant, fungal, bacterial, or viral protein) encoded by a reptilian, avian, amphibian, plant, fungal, bacterial, or viral gene that is homologous to an ETaG or NETag identified in an organism comprising a biosynthetic gene cluster (BGC) that produces the secondary metabolite.

In some instances, the methods of the present disclosure may further comprise performing an in vivo assay, for example, an assay to detect or measure an activity (e.g., a receptor binding activity, an enzyme activation activity, an enzyme inhibition activity, an intracellular signaling pathway activity, a disease response, etc.) of a secondary metabolite (or analog thereof) on a mammalian (e.g., human) protein encoded by a mammalian (e.g., human) gene that is homologous to an ETaG or NETaG identified in an organism comprising a biosynthetic gene cluster (BGC) that produces the secondary metabolite. In some instances, the methods may further comprise performing an in vivo assay to detect or measure an activity (e.g., a receptor binding activity, an enzyme activation activity, an enzyme inhibition activity, an intracellular signaling pathway activity, a disease response, etc.) of a secondary metabolite (or analog thereof) on a protein (e.g., a reptilian, avian, amphibian, plant, fungal, bacterial, or viral protein) encoded by a reptilian, avian, amphibian, plant, fungal, bacterial, or viral gene that is homologous to an ETaG or NETaG identified in an organism comprising a biosynthetic gene cluster (BGC) that produces the secondary metabolite.

In some instances, the methods of the present disclosure may be used, for example, for identifying and/or characterizing a mammalian (e.g., human) target of a secondary metabolite (or analog thereof) produced by a BGC. In some instances, the methods of the present disclosure may be used for identifying and/or characterizing a reptilian, avian, amphibian, plant, fungal, bacterial, viral target of a secondary metabolite (or analog thereof) produced by a BGC, or a target from any other organism.

In some instances, the methods of the present disclosure may be used, for example, for drug discovery activities, e.g., to identify small molecule modulators of a mammalian (e.g., human) target gene. In some instances, the methods of the present disclosure may be used to identify small molecule modulators of a reptilian target gene, an avian target gene, an amphibian target gene, a plant target gene, a fungal target gene, a bacterial target gene, a viral target gene, or a target gene from any other organism.

In some instances, the secondary metabolite is a product of enzymes encoded by the BGC or a salt thereof, including an unnatural salt. In some instances, the secondary metabolite or analog thereof is an analog of a product of enzymes encoded by the BGC, e.g., a small molecule compound having the same core structure as the secondary metabolite, or a salt thereof.

In some instances, the present disclosure provides methods for modulating a human target (or a target from another organism), comprising: providing a secondary metabolite produced by enzymes encoded by a BGC, or an analog thereof, wherein the human target (or a nucleic acid sequence encoding the human target) is homologous to an ETaG or NETaG that is associated with the BGC as determined using any one of the methods described herein.

In some instances, the present disclosure provides methods for treating a condition, disorder, or disease associated with a human target (or a target from another organism), comprising administering to a subject susceptible to, or suffering therefrom, a secondary metabolite produced by enzymes encoded by a BGC, or an analog thereof, wherein the human target (or a nucleic acid sequence encoding the human target) is homologous to an ETaG or NETaG that is associated with the BGC as determined using any one of the methods described herein.

In some instances, the secondary metabolite is produced by a fungus. In some instances, the secondary metabolite is acyclic. In some instances, the secondary metabolite is a polyketide. In some instances, the secondary metabolite is a terpene compound. In some instances, the secondary metabolite is a non-ribosomally synthesized peptide.

In some instances, an analog of a substance (e.g., secondary metabolite) that shares one or more particular structural features, elements, components, or moieties with a reference substance. Typically, an analog shows significant structural similarity with the reference substance, for example sharing a core or consensus structure, but also differs in certain discrete ways. In some instances, an analog is a substance that can be generated from the reference substance, e.g., by chemical manipulation of the reference substance. In some instances, an analog is a substance that can be generated through performance of a synthetic process substantially similar to (e.g., sharing a plurality of steps with) one that generates the reference substance. In some instances, an analog is or can be generated through performance of a synthetic process different from that used to generate the reference substance. In some instances, an analog of a substance is the substance being substituted at one or more of its substitutable positions.

In some instances, an analog of a product comprises the structural core of a product. In some instances, a biosynthetic product is cyclic, e.g., monocyclic, bicyclic, or polycyclic, and the structural core of the product is or comprises the monocyclic, bicyclic, or polycyclic ring system. In some instances, the structural core of the product comprises one ring of the bicyclic or polycyclic ring system of the product. In some instances, a product is or comprises a polypeptide, and a structural core is the backbone of the polypeptide. In some instances, a product is or comprises a polyketide, and a structural core is the backbone of the polyketide. In some instances, an analog is a substituted biosynthetic product comprising one or more suitable substituents.

### Systems for Identifying Gene Networks Containing Functionally-Related Genes

Also disclosed herein are systems designed to implement any of the disclosed methods for identifying gene networks (e.g., BGCs) containing sets of functionally-related genes. The systems may comprise, for example, one or more processors, and a memory unit communicatively coupled to the one or more processors and configured to store instructions that, when executed by the one or more processors, cause the system to receive a selection of genomes for analysis as input, wherein the selection of genomes comprises a plurality of related genomes; identify clusters of orthologous genes (COGs) in the plurality of related genomes; determine a pairwise co-evolution metric, a pairwise co-regulation metric, a pairwise co-occurrence metric, a pairwise co-localization metric, or any combination thereof, for the identified COGs; determine pairwise functional association scores for the identified COGs based on the determined pairwise co-evolution metrics, pairwise co-regulation metrics, pairwise co-occurrence metrics, pairwise co-localization metrics, or any combination thereof; cluster the identified COGs according to their pairwise functional association scores to group functionally-related COGs; and output, based on a functional enrichment analysis performed on at least one COG cluster to identify COG clusters that are enriched for genes in a specific functional category, a determination that a COG cluster is a network of functionally-related genes in the specific functional category.

### Computer Processors and Systems

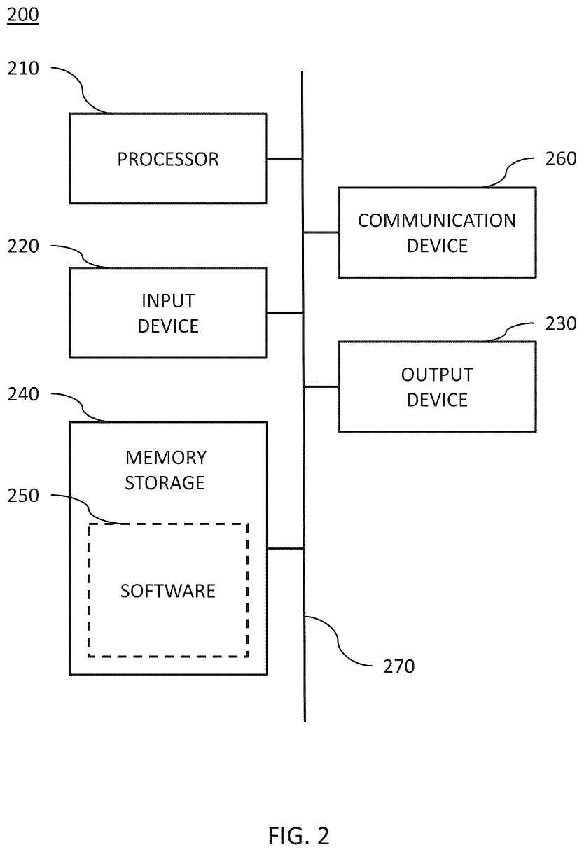

FIG. 2 illustrates an example of a computing device in accordance with one or more examples of the disclosure. Device 200 can be a host computer connected to a network. Device 200 can be a client computer or a server. As shown in FIG. 2, device 200 can be any suitable type of microprocessor-based device, such as a personal computer, workstation, server, or handheld computing device (portable electronic device), such as a phone or tablet. The device can include, for example, one or more of processor 210, input device 220, output device 230, storage 240, and communication device 260. Input device 220 and output device 230 can generally correspond to those described above, and they can either be connectable or integrated with the computer.

Input device 220 can be any suitable device that provides input, such as a touch screen, keyboard or keypad, mouse, or voice-recognition device. Output device 230 can be any suitable device that provides output, such as a touch screen, haptics device, or speaker.

Storage 240 can be any suitable device that provides storage, such as an electrical, magnetic, or optical memory including a RAM, cache, hard drive, or removable storage disk. Communication device 260 can include any suitable device capable of transmitting and receiving signals over a network, such as a network interface chip or device. The components of the computer can be connected in any suitable manner, such as via a physical bus 270 or wirelessly.

Software 250, which can be stored in memory/storage 240 and executed by processor 210, can include, for example, the programming that embodies the functionality of the present disclosure (e.g., as embodied in the devices described above).

Software 250 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 240, that can contain or store programming for use by or in connection with an instruction execution system, apparatus, or device.

Software 250 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate, or transport programming for use by or in connection with an instruction execution system, apparatus, or device. The transport readable medium can include, but is not limited to, an electronic, magnetic, optical, electromagnetic, or infrared wired or wireless propagation medium.

Device 200 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 200 can implement any operating system suitable for operating on the network. Software 350 can be written in any suitable programming language, such as C, C++, Java, or Python. In various embodiments, application software embodying the functionality of the present disclosure can be deployed in different configurations, such as in a client/server arrangement or through a web browser as a web-based application or web service, for example.

### EXAMPLES

### Example 1—Benchmarking on Known and Predicted BGCs

FIG. 3 provides a non-limiting schematic illustration of a pipeline for BGC discovery and functional assignment based on the methods described elsewhere herein. Process 300 begins with selection of a set of related genomes at step 302, followed by the identification of clusters of orthologous genes (COGs) at step 304, co-occurrence analysis at step 306, co-localization analysis at step 308, co-regulation analysis at step 310, co-evolution analysis at step 312, and the identification of candidate sets of functionally-related gene loci (i.e., candidate gene networks) at step 314. A statistical analysis (and, optionally, other post-processing steps) is performed at step 316 to identify a specific type of gene network, e.g., biosynthetic gene clusters (BGCs) at step 318.

To evaluate the approach described above, closely related fungal genomes known to possess BGCs for atpenin, cladosporin, citreoviridin, cyclosporin, restricticin or xanthocillin were identified. For each BGC type, 20 genomes showing ˜90% genome identity were selected. In addition, 6 very distantly related genomes (˜60% identical to the target set) were also selected as an outgroup. The best BLAST hits of the complement of proteins from all 26 genomes were computed and the pairwise percent identities obtained used as input for MCL clustering to group orthologous proteins (or the associate genes) in COGs. COGs containing proteins from all 20 target genomes and 6 outgroup genomes were considered to be part of the core genomes of these species and thus unlikely to play a role in secondary metabolism. These COGs were thus excluded from downstream analysis, speeding up the process.

Next, colocalization analysis was conducted by computing the average proximity score between all pairs of COGs as described elsewhere herein. This pairwise matrix was then used to cluster the COGs using MCL into cluster of colocalized (syntenous) COGs. These represent loci whose gene composition is conserved between all or a subset of the 20 target genomes considered for the analysis for each target. These syntenous COGs then served as the starting point for computing the other metrics used for delineating BGCs.

To evaluate co-regulation, the motif similarity score was computed (as described elsewhere herein) by first extracting the intergenic regions of all genes within the syntenous COGs and using this as input for de novo motif detection, with MEME, against a background distribution generated using all intergenic sequences from all genes across the target species. If a significant motif was detected, and at least one of the genes within a COG had a significant match to the motif in its intergenic region the COG was kept as member of the syntenous COG, or else the COG was dropped. This resulted in the generation of syntenous co-regulated COGs.

To evaluate co-evolution (as described elsewhere herein), for each syntenous COG all protein sequences within a COG were aligned using MAFFT and trimmed using trimAI to remove gaps. The pairwise percent identities were then computed between all gene pairs within a COG. To estimate coevolution, the Pearson correlation coefficient was computed from the percent identities of between all pairs on COGs in a synthenous COG for matching genome pairs. The pairwise matrix generated was filtered for correlation coefficients ≤0.9 and then used to cluster the COGs using MCL. Only the primary cluster of co-evolving COGs was retained.

To determine which of the identified loci are candidate BGCs, the syntenous COG information was first converted to gene-level clusters for each of the 20 target species used in the analysis. Each gene-level cluster was then evaluated for enrichment of PFAM domains known to occur within known BGCs using a hypergeometric test. Clusters having p-value ≤0.01 were considered candidate BGCs.

To evaluate the performance of the pipeline described above, three metrics were computed:
i. Recall of target cluster genes (i.e., the fraction of known cluster genes identified)

$$\frac{True Positive}{True Positive + False Negative}$$

ii. Precision of target cluster genes (i.e., the fraction of identified genes that are known target cluster genes)

$$\frac{True Positive}{True Positive + False Positive}$$

iii. Recall of all core synthase containing (antiSMASH predicted) clusters (to evaluate global performance)

$$\frac{True Positive ( identified antiSMASH clusters )}{True Positive + False Negative ( miss antiSMASH clusters )}$$

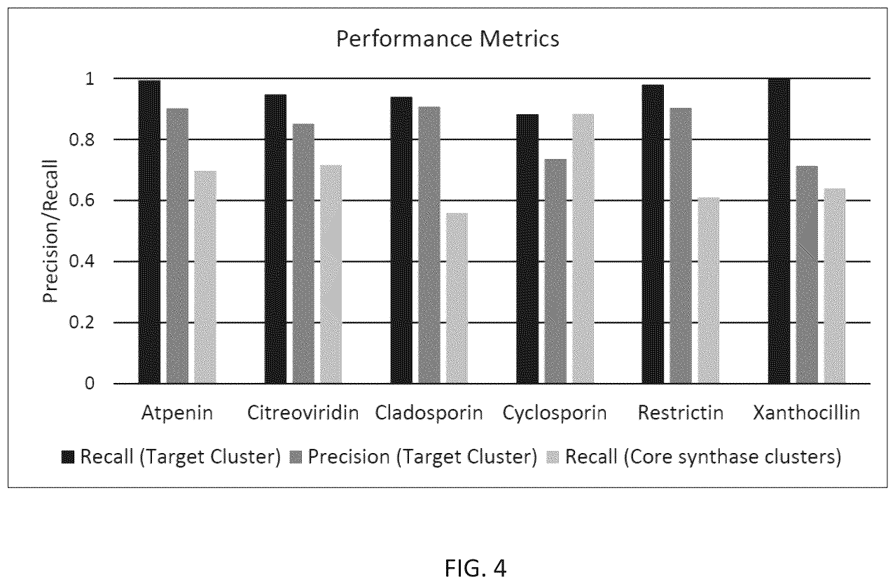

The above described BGC identification pipeline achieved a high degree of recall of the target cluster genes (i.e., fraction of genes known to be involved in the biosynthesis of the target molecule, for instance cyclosporin, that were captured). Recall for the 6 evaluated target BGCs ranged between 0.88 and 1, with an average of 0.95±0.04 (FIG. 4). In addition, the precision of this approach (i.e., fraction of predicted genes that are true target genes known to be involved in the biosynthesis of the target molecule), was also determined to be very high and ranged between 0.71 and 0.91 with an average precision of 0.83±0.09 (FIG. 4). Thus overall, the proposed pipeline performed well for identification of specific BGC genes with an overall F-score of ˜0.89.

To evaluate the performance of the pipeline more globally, its predictions were compared to the complement of BGCs predicted by the antiSMASH (Blin, et al. (2021), ibid.) (i.e., core synthase containing BGCs). Based on this comparison, we observed that our BGC detection approach was able to detect on average ˜70% of the antiSMASH predicted clusters across the 120 genomes selected for our analysis (FIG. 4). Overall, these results indicate that our approach can detect clusters with their appropriate boundaries (with only one or two additional or missing genes), while still performing well globally.

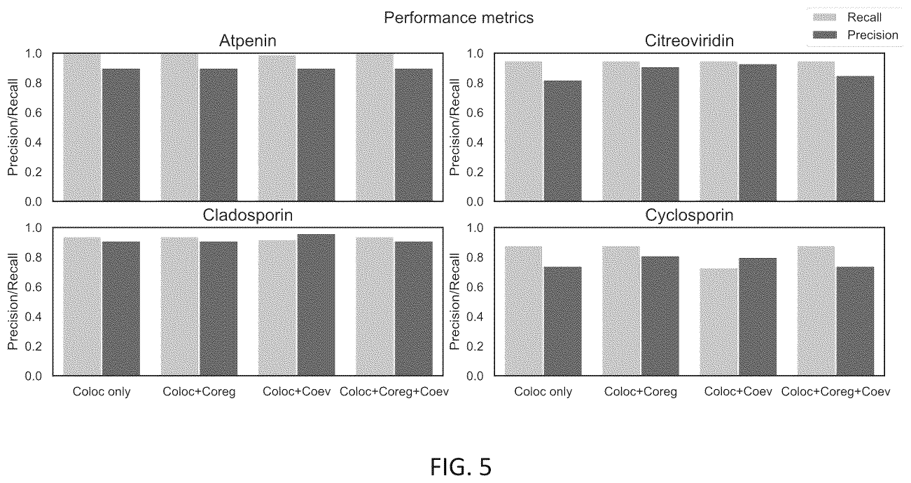

To assess, how each component of our pipeline improves predictive performance, we evaluated the use of colocalization in isolation or in combination with coregulation and coevolution (FIG. 5). Here we see that Recall of the target cluster genes is generally unimpacted by application of coregulation or coevolution, except in the case cyclosporin where recall drops from 0.88 to 0.73 (FIG. 5). This might be expected as the Recall values were already very high. On the other hand, precision is generally improved by application of coregulation and/or coevolution information for all targets except Atpenin, where the precision was already very high using colocalization information alone. These results indicate that depending on the cluster, incorporation of orthogonal sources of genomic data can a substantially improve the ability to accurately predict the target cluster genes.

Finally, one of the key advantages of our proposed pipeline is its ability to detect BGCs independent of core synthases, unlike many of the other BGC detection algorithms currently available, such as antiSMASH. To validate this, we included the BGC for Xanthocillin, which lacks a core synthase, in our benchmarking test set. Given that this BGC has no canonical core synthase it is not picked up by algorithms such as antiSMASH. However, as illustrated in FIG. 4, our proposed pipeline can identify this cluster with a similar degree of precision (0.71) and recall (1.0) as core synthase containing clusters. This indicates our pipeline is a more comprehensive tool for BGC detection than the current state of the art.

### EXEMPLARY EMBODIMENTS

Among the provided embodiments are:
1. A computer-implemented method for identification of networks of functionally-related genes, the method comprising:
   receiving a selection of genomes for analysis as input, wherein the selection of genomes comprises a plurality of related genomes;
   identifying clusters of orthologous genes (COGs) in the plurality of related genomes;
   determining a pairwise co-evolution metric, a pairwise co-regulation metric, a pairwise co-occurrence metric, a pairwise co-localization metric, or any combination thereof, for the identified COGs;
   determining pairwise functional association scores for the identified COGs based on the determined pairwise co-evolution metrics, pairwise co-regulation metrics, pairwise co-occurrence metrics, pairwise co-localization metrics, or any combination thereof;
   clustering the identified COGs according to their pairwise functional association scores to group functionally-related COGs; and
   outputting, based on a functional enrichment analysis performed on at least one COG cluster to identify COG clusters that are enriched for genes in a specific functional category, a determination that a COG cluster is a network of functionally-related genes in the specific functional category.
2. The computer-implemented method of embodiment 1, wherein the functional enrichment analysis does not require identification of a gene known to be associated with gene networks of a specific functional category.
3. The computer-implemented method of embodiment 1 or embodiment 2, wherein the functional enrichment analysis comprises testing for enrichment of genes in functional categories known to be associated with biosynthetic gene clusters (BGCs), thereby identifying those COG clusters as putative BGCs.
4. The computer-implemented method of embodiment 3, wherein the functional categories known to be associated with BGCs comprise gene ontology terms or KEGG pathways known to be associated with BGCs.
5. The computer-implemented method of embodiment 4, wherein the gene ontology terms known to be associated with BGCs comprise GO:0019748 (secondary metabolic process), GO:0044550 (secondary metabolite biosynthetic process), GO:0030639 (polyketide biosynthetic process), GO:0030638 (polyketide metabolic process), GO:0043455 (regulation of secondary metabolic process), GO:1900539 (fumonisin metabolic process), or any combination thereof.
6. The computer-implemented method of embodiment 4, wherein the KEGG pathways known to be associated with BGCs comprise M00778 (Type II polyketide backbone biosynthesis) or M00095 (C5 isoprenoid biosynthesis, mevalonate pathway), M00937 (Aflatoxin biosynthesis), M00893 (Lovastatin biosynthesis), or any combination thereof.
7. The computer-implemented method of any one of embodiments 1 to 6, wherein the functional enrichment analysis comprises testing for enrichment of protein domain representations known to be associated with biosynthetic gene clusters (BGCs), thereby identifying those COG clusters as putative BGCs.
8. The computer-implemented method of embodiment 7, wherein the protein domain representations known to be associated with BGCs comprise PFAM domain representations, Conserved Domain Database (CDD) domain representations, or TIGRFAM domain representations known to be associated with BGCs.
9. The computer-implemented method of any one of embodiments 2 to 8, further comprising identifying putative targets for a secondary metabolite synthesized by a putative BGC by:
   identifying protein sequences that are not components of known BGCs;
   determining pairwise functional association scores for the identified protein sequences and the putative BGC; and
   identifying putative targets for the secondary metabolite based on a comparison of the pairwise functional association scores to a first predetermined threshold.
10. The computer-implemented method of embodiment 9, wherein the pairwise functional associate score comprises a co-regulation score, and the first predetermined threshold for the co-regulation score corresponds to a p-value of less than or equal to 0.05.
11. The computer-implemented method of embodiment 9, wherein the pairwise functional association score comprises a co-evolution score, and the first predetermined threshold for the co-evolution score corresponds to a co-evolution score value of greater than or equal to 0.7.
12. The computer-implemented method of embodiment 9, wherein the pairwise functional association score comprises a co-occurrence score, and the first predetermined threshold for the co-occurrence score corresponds to a co-occurrence score of greater than or equal to 0.5.
13. The computer-implemented method of any one of embodiments 2 to 12, wherein identification of a putative BGC does not require identification of an associated core synthase.
14. The computer-implemented method of any one of embodiments 1 to 13, wherein the plurality of related genomes comprise fungal, bacterial, or plant genomes.
15. The computer-implemented method of any one of embodiments 1 to 14, wherein identifying COGs comprises identifying orthologous genes in the plurality of related genomes as bidirectional best hits using BLAST, followed by clustering of the identified orthologous genes.
16. The computer-implemented method of any one of embodiments 1 to 15, wherein identifying COGs comprises identifying orthologous genes in the plurality of related genomes using orthoMCL or orthoFinder.
17. The computer-implemented method of any one of embodiments 1 to 16, wherein determining a pairwise co-evolution metric for COGs comprises:
   computing a percent identity between each pair of protein sequences within each COG of a pair of COGs to identify shared protein sequences;
   computing a Pearson's correlation coefficient for each pair of COGs that include a specified minimum number of shared protein sequences to estimate a rate of co-evolution;
   filtering the COGs by excluding COGs for which the pairwise Pearson's correlation coefficient is less than a second predetermined threshold and clustering the remaining COGs according to the estimated rates of co-evolution; and
   performing a functional enrichment analysis to exclude clusters of COGS that are enriched for essential metabolic functional categories.
18. The computer-implemented method of embodiment 17, wherein the second predetermined threshold corresponds to a Pearson's correlation coefficient value of 0.7, 0.8, 0.9, 0.95, 0.98, or 0.99.
19. The computer-implemented method of embodiment 17 or embodiment 18, wherein clustering the remaining COGs according to the estimated rates of co-evolution comprises use of a Markov clustering (MCL) or hierarchical clustering algorithm.
20. The computer-implemented method of any one of embodiments 1 to 19, wherein determining a pairwise co-regulation metric for COGs comprises:
   extracting intergenic regions within each COG;
   performing a de novo detection of sequence motifs within the extracted intergenic regions to identify putative cis-regulatory elements or transcription factor binding sites (TFBS);
   comparing the putative cis-regulatory elements or TFBS identified for each COG to those identified across all other COGs to determine pairwise motif similarity scores between COGs;
   filtering the COGs to exclude COGs for which pairwise motif similarity scores have a p-value of less than or equal to a third predetermined threshold; and
   clustering the filtered COGs based on the pairwise motif similarity scores to identify co-regulated COG clusters.
21. The computer-implemented method of embodiment 20, wherein the third predetermined threshold corresponds to a p-value of 0.05.
22. The computer-implemented method of embodiment 20, wherein the third predetermined threshold corresponds to a p-value of 0.01.
23. The computer-implemented method of any one of embodiments 20 to 22, wherein clustering the remaining COGs according to the motif similarity scores comprises use of a Markov clustering (MCL) or hierarchical clustering algorithm.
24. The computer-implemented method of any one of embodiments 1 to 23, wherein determining a pairwise co-occurrence metric for COGs comprises:
   computing a Jaccard index for each pair of COGs (COG A and COG B) based on a relationship:

Co-occurrence score=(|A∩B|)/(|A∪B|)
filtering the COGs to exclude COGs for which pairwise co-occurrence scores have a value of less than a fourth predetermined threshold; and
clustering the filtered COGs based on the pairwise co-occurrence scores to identify co-occurring COG clusters.
25. The computer-implemented method of embodiment 24, wherein the fourth predetermined threshold corresponds to a pairwise co-occurrence score value of 0.3, 0.4, 0.5, 0.6, 0.7, 0.8, or 0.9.
26. The computer-implemented method of embodiment 24 or embodiment 25, wherein clustering the remaining COGs according to the co-occurrence scores comprises use of a Markov clustering (MCL) or hierarchical clustering algorithm.
27. The computer-implemented method of any one of embodiments 1 to 26, wherein determining a pairwise co-localization metric for COGs comprises:
   computing a proximity score for each pair of corresponding gene sequences in a pair of COGS (COG A and COG B) based on a relationship:

$$Proximity score = 1 / ( 1 + \# of interspacing genes$$

averaging to determine the co-localization metric for each pair of COGs; and
clustering the COGs based on the averaged proximity score to identify co-localized COG clusters.
28. The computer-implemented method of embodiment 27, wherein clustering of COGs according to the averaged proximity score comprises use of a Markov clustering (MCL) or hierarchical clustering algorithm.
29. The computer-implemented method of embodiment 27 or embodiment 28, wherein the clustering is performed using a Markov clustering (MCL) algorithm and an MCL inflation value ranging from 1.5 to 5.0.
30. The computer-implemented method of any one of embodiments 1 to 29, wherein the pairwise functional association score for the identified COGs is based on addition of the determined pairwise co-evolution metrics, pairwise co-regulation metrics, pairwise co-occurrence metrics, pairwise co-localization metrics, or any combination thereof.
31. The computer-implemented method of any one of embodiments 1 to 30, wherein clustering the identified COGs according to their pairwise functional association scores comprises the use of a use of a Markov clustering (MCL) or hierarchical clustering algorithm.
32. The computer-implemented method of any one of embodiments 1 to 31, further comprising determining a horizontal gene transfer metric based on computing a codon adaptation index (CAI) or a dinucleotide signature dissimilarity index (DSDI).
33. The computer-implemented method of embodiment 32, wherein the horizontal gene transfer metric is used to further refine clustering of co-localized, co-occurring and/or co-evolving COGs.
34. The computer-implemented method of embodiment 32, wherein the horizontal gene transfer metric is used as part of a post-processing step to retrieve nearby horizontally transferred genes missed in upstream clustering steps.
35. The computer-implemented method of any one of embodiments 3 to 34, further comprising evaluating a gene identified as belonging to the putative BGC to determine if it is a resistance gene.
36. The computer-implemented method of embodiment 35, wherein the resistance gene is an embedded target gene (ETaG) or a non-embedded target gene (NETaG).
37. The computer-implemented method of embodiment 35 or embodiment 36, further comprising performing an in vitro assay to test a secondary metabolite produced by the putative BGC for activity against a resistance gene homolog, or protein encoded thereby, identified in a target genome.
38. The computer-implemented method of any one of embodiments 35 to 37, further comprising performing an in vivo assay to test a secondary metabolite produced by the putative BGC for activity against a resistance gene homolog, or protein encoded thereby, identified in a target genome.
39. The computer-implemented method of embodiment 37 or embodiment 38, wherein the target genome comprises a mammalian genome, a human genome, an avian genome, a reptilian genome, an amphibian genome, a plant genome, a fungal genome, a bacterial genome, or a viral genome.
40. A system comprising:
   one or more processors; and
   a memory communicatively coupled to the one or more processors and configured to store instructions that, when executed by the one or more processors, cause the system to:
      receive a selection of genomes for analysis as input, wherein the selection of genomes comprises a plurality of related genomes;
      identify clusters of orthologous genes (COGs) in the plurality of related genomes;
      determine a pairwise co-evolution metric, a pairwise co-regulation metric, a pairwise co-occurrence metric, a pairwise co-localization metric, or any combination thereof, for the identified COGs;
      determine pairwise functional association scores for the identified COGs based on the determined pairwise co-evolution metrics, pairwise co-regulation metrics, pairwise co-occurrence metrics, pairwise co-localization metrics, or any combination thereof;
      cluster the identified COGs according to their pairwise functional association scores to group functionally-related COGs; and
      output, based on a functional enrichment analysis performed on at least one COG cluster to identify COG clusters that are enriched for genes in a specific functional category, a determination that a COG cluster is a network of functionally-related genes in the specific functional category.
41. The system of embodiment 40, wherein the functional enrichment analysis does not require identification of a gene known to be associated with gene networks of a specific functional category.
42. The system of embodiment 40 or embodiment 41, wherein the functional enrichment analysis comprises testing for enrichment of genes in functional categories known to be associated with biosynthetic gene clusters (BGCs), thereby identifying those COG clusters as putative BGCs.
43. The system of embodiment 42, wherein the functional categories known to be associated with BGCs comprise gene ontology terms or KEGG pathways known to be associated with BGCs.
44. The system of any one of embodiments 40 to 43, wherein the functional enrichment analysis comprises testing for enrichment of protein domain representations known to be associated with biosynthetic gene clusters (BGCs), thereby identifying those COG clusters as putative BGCs.
45. The system of embodiment 44, wherein the protein domain representations known to be associated with BGCs comprise PFAM domain representations, Conserved Domain Database (CDD) domain representations, or TIGRFAM domain representations known to be associated with BGCs.
46. The system of any one of embodiments 42 to 45, further comprising instructions for identifying putative targets for a secondary metabolite synthesized by a putative BGC by:
   identifying protein sequences that are not components of known BGCs;
   determining pairwise functional association scores for the identified protein sequences and the putative BGC; and
   identifying putative targets for the secondary metabolite based on a comparison of the pairwise functional association scores to a first predetermined threshold.
47. The system of embodiment 46, wherein the pairwise functional associate score comprises a co-regulation score, and the first predetermined threshold for the co-regulation score corresponds to a p-value of less than or equal to 0.05.
48. The system of embodiment 46, wherein the pairwise functional association score comprises a co-evolution score, and the first predetermined threshold for the co-evolution score corresponds to a co-evolution score value of greater than or equal to 0.7.
49. The system of embodiment 46, wherein the pairwise functional association score comprises a co-occurrence score, and the first predetermined threshold for the co-occurrence score corresponds to a co-occurrence score of greater than or equal to 0.5.
50. The system of any one of embodiments 42 to 49, wherein identification of a putative BGC does not require identification of an associated core synthase.
51. A non-transitory computer-readable medium storing one or more programs, the one or more programs comprising instructions, which when executed by one or more processors of a system, cause the system to:
   receive a selection of genomes for analysis as input, wherein the selection of genomes comprises a plurality of related genomes;
   identify clusters of orthologous genes (COGs) in the plurality of related genomes;
   determine a pairwise co-evolution metric, a pairwise co-regulation metric, a pairwise co-occurrence metric, a pairwise co-localization metric, or any combination thereof, for the identified COGs;
   determine pairwise functional association scores for the identified COGs based on the determined pairwise co-evolution metrics, pairwise co-regulation metrics, pairwise co-occurrence metrics, pairwise co-localization metrics, or any combination thereof;
   cluster the identified COGs according to their pairwise functional association scores to group functionally-related COGs; and
   output, based on a functional enrichment analysis performed on at least one COG cluster to identify COG clusters that are enriched for genes in a specific functional category, a determination that a COG cluster is a network of functionally-related genes in the specific functional category.
52. The non-transitory computer-readable medium of embodiment 51, wherein the functional enrichment analysis does not require identification of a gene known to be associated with gene networks of a specific functional category.
53. The non-transitory computer-readable medium of embodiment 51 or embodiment 52, wherein the functional enrichment analysis comprises testing for enrichment of genes in functional categories known to be associated with biosynthetic gene clusters (BGCs), thereby identifying those COG clusters as putative BGCs.
54. The non-transitory computer-readable medium of embodiment 43, wherein the functional categories known to be associated with BGCs comprise gene ontology terms or KEGG pathways known to be associated with BGCs.
55. The non-transitory computer-readable medium of any one of embodiments 51 to 54, wherein the functional enrichment analysis comprises testing for enrichment of protein domain representations known to be associated with biosynthetic gene clusters (BGCs), thereby identifying those COG clusters as putative BGCs.
56. The non-transitory computer-readable medium of embodiment 55, wherein the protein domain representations known to be associated with BGCs comprise PFAM domain representations, Conserved Domain Database (CDD) domain representations, or TIGRFAM domain representations known to be associated with BGCs.
57. The non-transitory computer-readable medium of any one of embodiments 53 to 56, further comprising instructions for identifying putative targets for a secondary metabolite synthesized by a putative BGC by:
   identifying protein sequences that are not components of known BGCs;
   determining pairwise functional association scores for the identified protein sequences and the putative BGC; and
   identifying putative targets for the secondary metabolite based on a comparison of the pairwise functional association scores to a first predetermined threshold.
58. The non-transitory computer-readable medium of embodiment 57, wherein the pairwise functional associate score comprises a co-regulation score, and the first predetermined threshold for the co-regulation score corresponds to a p-value of less than or equal to 0.05.
59. The non-transitory computer-readable medium of embodiment 57, wherein the pairwise functional association score comprises a co-evolution score, and the first predetermined threshold for the co-evolution score corresponds to a co-evolution score value of greater than or equal to 0.7.
60. The non-transitory computer-readable medium of embodiment 57, wherein the pairwise functional association score comprises a co-occurrence score, and the first predetermined threshold for the co-occurrence score corresponds to a co-occurrence score of greater than or equal to 0.5.
61. The non-transitory computer-readable medium of any one of embodiments 55 to 60, wherein identification of a putative BGC does not require identification of an associated core synthase.

It should be understood from the foregoing that, while particular implementations of the disclosed methods and systems have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

1. A computer-implemented method for identification of networks of functionally-related genes, the method comprising:
receiving a selection of genomes for analysis as input, wherein the selection of genomes comprises a plurality of related genomes;
identifying clusters of orthologous genes (COGs) in the plurality of related genomes;
determining a pairwise co-evolution metric, a pairwise co-regulation metric, a pairwise co-occurrence metric, a pairwise co-localization metric, or any combination thereof, for the identified COGs;
determining pairwise functional association scores for the identified COGs based on the determined pairwise co-evolution metrics, pairwise co-regulation metrics, pairwise co-occurrence metrics, pairwise co-localization metrics, or any combination thereof;
clustering the identified COGs according to their pairwise functional association scores to group functionally-related COGs; and
outputting, based on a functional enrichment analysis performed on at least one COG cluster to identify COG clusters that are enriched for genes in a specific functional category, a determination that a COG cluster is a network of functionally-related genes in the specific functional category.

2. The computer-implemented method of claim 1, wherein the functional enrichment analysis does not require identification of a gene known to be associated with gene networks of a specific functional category.

3. The computer-implemented method of claim 1, wherein the functional enrichment analysis comprises testing for enrichment of genes in functional categories known to be associated with biosynthetic gene clusters (BGCs), thereby identifying those COG clusters as putative BGCs.

4. The computer-implemented method of claim 3, wherein the functional categories known to be associated with BGCs comprise gene ontology terms or KEGG pathways known to be associated with BGCs.

5. The computer-implemented method of claim 4, wherein the gene ontology terms known to be associated with BGCs comprise GO:0019748 (secondary metabolic process), GO:0044550 (secondary metabolite biosynthetic process), GO:0030639 (polyketide biosynthetic process), GO:0030638 (polyketide metabolic process), GO:0043455 (regulation of secondary metabolic process), GO:1900539 (fumonisin metabolic process), or any combination thereof.

6. The computer-implemented method of claim 4, wherein the KEGG pathways known to be associated with BGCs comprise M00778 (Type II polyketide backbone biosynthesis) or M00095 (C5 isoprenoid biosynthesis, mevalonate pathway), M00937 (Aflatoxin biosynthesis), M00893 (Lovastatin biosynthesis), or any combination thereof.

7. The computer-implemented method of claim 1, wherein the functional enrichment analysis comprises testing for enrichment of protein domain representations known to be associated with biosynthetic gene clusters (BGCs), thereby identifying those COG clusters as putative BGCs.

8. The computer-implemented method of claim 7, wherein the protein domain representations known to be associated with BGCs comprise PFAM domain representations, Conserved Domain Database (CDD) domain representations, or TIGRFAM domain representations known to be associated with BGCs.

9. The computer-implemented method of claim 2, further comprising identifying putative targets for a secondary metabolite synthesized by a putative BGC by:
identifying protein sequences that are not components of known BGCs;
determining pairwise functional association scores for the identified protein sequences and the putative BGC; and
identifying putative targets for the secondary metabolite based on a comparison of the pairwise functional association scores to a first predetermined threshold.

10. The computer-implemented method of claim 9, wherein the pairwise functional associate score comprises a co-regulation score, and the first predetermined threshold for the co-regulation score corresponds to a p-value of less than or equal to 0.05.

11. The computer-implemented method of claim 9, wherein the pairwise functional association score comprises a co-evolution score, and the first predetermined threshold for the co-evolution score corresponds to a co-evolution score value of greater than or equal to 0.7.

12. The computer-implemented method of claim 9, wherein the pairwise functional association score comprises a co-occurrence score, and the first predetermined threshold for the co-occurrence score corresponds to a co-occurrence score of greater than or equal to 0.5.

13. The computer-implemented method of claim 2, wherein identification of a putative BGC does not require identification of an associated core synthase.

14. The computer-implemented method of claim 1, wherein the plurality of related genomes comprise fungal, bacterial, or plant genomes.

15. The computer-implemented method of claim 1, wherein identifying COGs comprises identifying orthologous genes in the plurality of related genomes as bidirectional best hits using BLAST, followed by clustering of the identified orthologous genes.

16. The computer-implemented method of claim 1, wherein identifying COGs comprises identifying orthologous genes in the plurality of related genomes using orthoMCL or orthoFinder.

17. The computer-implemented method of claim 1, wherein determining a pairwise co-evolution metric for COGs comprises:
computing a percent identity between each pair of protein sequences within each COG of a pair of COGs to identify shared protein sequences;
computing a Pearson's correlation coefficient for each pair of COGs that include a specified minimum number of shared protein sequences to estimate a rate of co-evolution;
filtering the COGs by excluding COGs for which the pairwise Pearson's correlation coefficient is less than a second predetermined threshold and clustering the remaining COGs according to the estimated rates of co-evolution; and
performing a functional enrichment analysis to exclude clusters of COGS that are enriched for essential metabolic functional categories.

18. The computer-implemented method of claim 17, wherein the second predetermined threshold corresponds to a Pearson's correlation coefficient value of 0.7, 0.8, 0.9, 0.95, 0.98, or 0.99.

19. The computer-implemented method of claim 17, wherein clustering the remaining COGs according to the estimated rates of co-evolution comprises use of a Markov clustering (MCL) or hierarchical clustering algorithm.

20. The computer-implemented method of claim 1, wherein determining a pairwise co-regulation metric for COGs comprises:
extracting intergenic regions within each COG;
performing a de novo detection of sequence motifs within the extracted intergenic regions to identify putative cis-regulatory elements or transcription factor binding sites (TFBS);
comparing the putative cis-regulatory elements or TFBS identified for each COG to those identified across all other COGs to determine pairwise motif similarity scores between COGs;
filtering the COGs to exclude COGs for which pairwise motif similarity scores have a p-value of less than or equal to a third predetermined threshold; and
clustering the filtered COGs based on the pairwise motif similarity scores to identify co-regulated COG clusters.

21. The computer-implemented method of claim 20, wherein the third predetermined threshold corresponds to a p-value of 0.05.

22. The computer-implemented method of claim 20, wherein the third predetermined threshold corresponds to a p-value of 0.01.

23. The computer-implemented method of claim 20, wherein clustering the remaining COGs according to the motif similarity scores comprises use of a Markov clustering (MCL) or hierarchical clustering algorithm.

24. The computer-implemented method of claim 1, wherein determining a pairwise co-occurrence metric for COGs comprises:
computing a Jaccard index for each pair of COGs (COG A and COG B) based on a relationship:
Co-occurrence score=(|A∩B|)/(|A∪B|)
filtering the COGs to exclude COGs for which pairwise co-occurrence scores have a value of less than a fourth predetermined threshold; and
clustering the filtered COGs based on the pairwise co-occurrence scores to identify co-occurring COG clusters.

25. The computer-implemented method of claim 24, wherein the fourth predetermined threshold corresponds to a pairwise co-occurrence score value of 0.3, 0.4, 0.5, 0.6, 0.7, 0.8, or 0.9.

26. The computer-implemented method of claim 24, wherein clustering the remaining COGs according to the co-occurrence scores comprises use of a Markov clustering (MCL) or hierarchical clustering algorithm.

27. The computer-implemented method of claim 1, wherein determining a pairwise co-localization metric for COGs comprises:
computing a proximity score for each pair of corresponding gene sequences in a pair of COGS (COG A and COG B) based on a relationship:
$$Proximity score = 1 / ( 1 + \# of interspacing genes$$
averaging to determine the co-localization metric for each pair of COGs; and
clustering the COGs based on the averaged proximity score to identify co-localized COG clusters.

28. The computer-implemented method of claim 27, wherein clustering of COGs according to the averaged proximity score comprises use of a Markov clustering (MCL) or hierarchical clustering algorithm.

29. The computer-implemented method of claim 27, wherein the clustering is performed using a Markov clustering (MCL) algorithm and an MCL inflation value ranging from 1.5 to 5.0.

30. The computer-implemented method of claim 1, wherein the pairwise functional association score for the identified COGs is based on addition of the determined pairwise co-evolution metrics, pairwise co-regulation metrics, pairwise co-occurrence metrics, pairwise co-localization metrics, or any combination thereof.

31. The computer-implemented method of claim 1, wherein clustering the identified COGs according to their pairwise functional association scores comprises the use of a use of a Markov clustering (MCL) or hierarchical clustering algorithm.

32. The computer-implemented method of claim 1, further comprising determining a horizontal gene transfer metric based on computing a codon adaptation index (CAI) or a dinucleotide signature dissimilarity index (DSDI).

33. The computer-implemented method of claim 32, wherein the horizontal gene transfer metric is used to further refine clustering of co-localized, co-occurring and/or co-evolving COGs.

34. The computer-implemented method of claim 32, wherein the horizontal gene transfer metric is used as part of a post-processing step to retrieve nearby horizontally transferred genes missed in upstream clustering steps.

35. The computer-implemented method of claim 3, further comprising evaluating a gene identified as belonging to the putative BGC to determine if it is a resistance gene.

36. The computer-implemented method of claim 35, wherein the resistance gene is an embedded target gene (ETaG) or a non-embedded target gene (NETaG).

37. The computer-implemented method of claim 35, further comprising performing an in vitro assay to test a secondary metabolite produced by the putative BGC for activity against a resistance gene homolog, or protein encoded thereby, identified in a target genome.

38. The computer-implemented method of claim 35, further comprising performing an in vivo assay to test a secondary metabolite produced by the putative BGC for activity against a resistance gene homolog, or protein encoded thereby, identified in a target genome.

39. The computer-implemented method of claim 38, wherein the target genome comprises a mammalian genome, a human genome, an avian genome, a reptilian genome, an amphibian genome, a plant genome, a fungal genome, a bacterial genome, or a viral genome.

40-61. (canceled)
